(19) United States
(12) Patent Application Publication
(10) Pub. No.: US 2024/0081656 A1
(43) Pub. Date: Mar. 14, 2024

(54) WEARABLE PHYSIOLOGICAL MONITORING SYSTEM

(71) Applicant: Masimo Corporation, Irvine, CA (US)

(72) Inventors: Chad A. DeJong, Los Angeles, CA (US); Stephen Scruggs, Newport Beach, CA (US); Ammar Al-Ali, San Juan Capistrano, CA (US)

(21) Appl. No.: 18/464,181

(22) Filed: Sep. 8, 2023

Related U.S. Application Data
(63) Continuation-in-part of application No. 29/911,847, filed on Sep. 8, 2023.
Application No. 29/911,847 is a continuation-in-part of application No. 29/879,161, filed on Jul. 3, 2023.
(60) Provisional application No. 63/511,830, filed on Jul. 3, 2023.
(60) Provisional application No. 63/387,048, filed on Dec. 12, 2022.
(60) Provisional application No. 63/386,474, filed on Dec. 7, 2022.
(60) Provisional application No. 63/375,191, filed on Sep. 9, 2022.

(51) Int. Cl. A61B 5/0205; A61B 5/00; A61B 5/1455; A61B 5/332

(52) U.S. Cl. CPC A61B 5/0205; A61B 5/0006; A61B 5/14552; A61B 5/332; A61B 5/6804; A61B 5/6831; A61B 2560/0443

(57) ABSTRACT

A wearable system can include an electronic device configured to measure one or more physiological parameters of a subject and a wearable device configured to cover and position the electronic device. The electronic device can comprise at least one light emitter and at least one light detector and is configured to measure at least a pulse oximetry measurement. The wearable device can comprise a body portion comprising a first side, a second side opposite the first side, a cavity, and an opening in the second side configured to allow the electronic device to be at least partially inserted through said opening and at least partially positioned within said cavity, and a securement portion connected to the body portion and configured to secure said body portion to the subject to prevent the electronic device from slipping or moving along a tissue site of the subject.

### INCORPORATION BY REFERENCE TO ANY PRIORITY APPLICATIONS

The present application claims priority to U.S. Provisional Application No. 63/375,191, filed Sep. 9, 2022, U.S. Provisional Application No. 63/386,474, filed Dec. 7, 2022, U.S. Provisional Application No. 63/387,048, filed Dec. 12, 2022, and U.S. Provisional Application No. 63/511,830, filed Jul. 3, 2023, U.S. Design patent application Ser. No. 29/879161, filed Jul. 3, 2023, and U.S. Design patent application Ser. No. 29/911847, filed Sep. 8, 2023. All of the above-listed applications and any and all other applications for which a foreign or domestic priority claim is identified in the Application Data Sheet as filed with the present application, are hereby incorporated by reference under 37 CFR 1.57, and form a part of this specification for all purposes.

### TECHNICAL FIELD

The present disclosure relates to a wearable physiological monitoring system incorporating an electronic device for measuring and/or monitoring a subject's physiological information including at least a pulse oximetry measurement.

### BACKGROUND

Pulse oximetry is a widely accepted noninvasive procedure for measuring the oxygen saturation level of arterial blood, an indicator of a person's oxygen supply. Pulse oximetry sensors generally include one or more light sources transmitting optical radiation into or reflecting off through a portion of the body. After attenuation by tissue and fluids of the portion of the body, one or more photodetection devices detect the attenuated light and output one or more detector signals responsive to the detected attenuated light.

### SUMMARY

Pulse oximetry sensors can be utilized for determination of a variety of physiological parameters and/or characteristics, including but not limited to oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), glucose, and/or otherwise, and the pulse oximetry sensor can be utilized for display on one or more electronic devices the foregoing parameters individually, in groups, in trends, as combinations, or as an overall wellness or other index.

A drawback to current pulse oximetry sensors is a need to be located near significant capillary beds on the body, including fingers, ears, toes, nose and forehead. Such locations are often inconvenient for monitoring a subject during normal activities, such as outside of a healthcare facility. Further, although measuring through motion oxygen saturation technology exists, it is directed to the healthcare facility context and is not reliable for normal routines, which include sporting activities or other significant daily movement. Accordingly, the present disclosure provides a wearable system which allows for measuring pulse oximetry at sparse capillary bed locations, including the wrist, lower arm, and/or upper arm. Such measurement locations can advantageously provide a wearable system for measuring pulse oximetry through everyday motion and higher exertion activities.

As described above, it can be advantageous to select a wrist, a lower arm, and/or an upper arm as a site for pulse oximetry. The present disclosure describes various implementations for wearable systems which secure to a subject (e.g., to a wrist, a lower arm, an upper arm, and/or an upper body of the subject) and employ pulse oximetry at a wrist, a lower arm, and/or an upper arm of the subject. Various implementations disclosed herein provide increased user comfort, increased user ergonomics, facilitate better sensor-skin contact and engagement, allow for everyday motion and/or higher exertion activities (e.g., sports or workout activities), and provide better stability in securement (e.g., to prevent the sensor from slipping or moving along a tissue site of the subject) in order to provide more accurate physiological parameter determination during everyday motion and/or higher exertion activities. Some implementations of the wearable systems disclosed herein include a wearable device configured to be secured to the subject and operably position an electronic device configured to measure at least a pulse oximetry measurement of the subject. The wearable devices described herein can include a body portion (which can also be referred to herein as a “main body”) configured to operably position the electronic device and a securement portion connected to the body portion configured to secure the body portion to the subject. In some implementations, the securement portion includes a strap, a band, or a garment. Such a garment can include a shirt, a jacket, a dress, an upper-body-worn garment, or any other garment. Advantageously, such wearable devices can allow the wearable systems described herein to monitor and/or measure one or more physiological parameters of the subject inconspicuously and/or hidden entirely from view by others (e.g., if the subject desires to hide and/or mask such monitoring and/or measuring). In some implementations, the wearable systems described herein advantageously provide a low-profile, non-bulky, and/or comfortable physiological monitoring and/or measuring system.

Some implementations of the wearable systems disclosed herein provide for a wearable system that can easily be adapted and/or customized to fit subjects with body parts (e.g., wrists, lower arms, upper arms, and/or upper bodies) of various sizes and/or shapes. For example, the wearable devices described herein can be adjustable, flexible, and/or stretchy to allow for a customized fit for the subject. Additionally, the wearable devices described herein can be provided in various sizes/shapes (e.g., small, medium, large, extra-large) to further enable a customized fit for the subject. Some implementations of the wearable systems disclosed herein can advantageously provide for a wearable system that is reusable and/or durable and/or have components that are reusable and/or durable (e.g., lasting weeks, months, and/or years). Some implementations of the wearable systems disclosed herein can provide for an electronic device that is reusable and/or durable. Additionally, some implementations of the wearable systems disclosed herein can provide for a wearable device that is reusable and/or durable. Alternatively, some implementations of the wearable systems disclosed herein can provide for a wearable device that is disposable. In some implementations, reusable components of the wearable systems described herein can be refurbished, reused, and/or reprocessed. Such reusable components can be combined with other new, refurbished, reused, and/or reprocessed components to form a portion or an entirety of a wearable system. In some implementations, such reusable components can be combined with disposable components to form a portion or an entirety of a wearable system.

While the wearable systems described herein are described as being adapted to secure to an upper body of a subject and/or portions thereof (e.g., wrist, lower arm, upper arm, upper body), such disclosure is not intended to be limiting. In some implementations, the wearable systems described herein can be adapted to secure to other portions of a subject, such as an ankle, a lower leg, an upper leg, a lower body of the subject, and/or a torso or mid portion of the subject's body. In such implementations, the securement portion of the wearable device of the wearable system can include a garment such as a sock, a pair of shorts, a pair of pants, athletic wear (e.g., compression clothing), a belt, or the like.

In some implementations, the wearable systems described herein can be configured to monitor and/or measure a physiological parameter of the subject in addition to a pulse oximetry measurement. For example, the electronic devices of the wearable systems described herein can include at least one ECG electrode and can be configured to measure at least an ECG measurement. As another example, the electronic devices of the of the wearable systems described herein can include at least one temperature sensor and can be configured to measure at least a temperature measurement. The electronic devices of the wearable systems described herein can include a communication module configured to facilitate wireless communication with a separate device, such as a mobile phone of the subject, for reporting and/or displaying any of the physiological measurements obtained by the wearable system.

Disclosed herein is a wearable system comprising an electronic device and a wearable device. The electronic device can be configured to measure one or more physiological parameters of a subject. The electronic device can comprise at least one light emitter and at least one light detector and can be configured to measure at least a pulse oximetry measurement. The wearable device can be configured to cover and position the electronic device. The wearable device can comprise: a body portion comprising a first side, a second side opposite the first side, a cavity, and an opening in the second side configured to allow the electronic device to be at least partially inserted through said opening and at least partially positioned within said cavity; and a securement portion connected to the body portion and configured to secure said body portion to the subject to prevent the electronic device from slipping or moving along a tissue site of the subject. Said second side of said body portion can be configured to face toward said tissue site of the subject when the wearable device is secured to the subject. Furthermore, the wearable device can cover the electronic device such that the electronic device is not visible when the securement portion secures the body portion to the subject.

In the above wearable system or in other implementations as described herein, one or more of the following features can also be provided. In some implementations, the electronic device further comprises: at least one ECG electrode and is configured to measure at least an ECG measurement; and a communication module configured to facilitate wireless communication with a separate device. In some implementations, the electronic device comprises a battery and a charge port, and the charge port is accessible for charging the battery of the electronic device when the electronic device is positioned at least partially within said cavity. In some implementations, said securement portion comprises a strap, a band, or a garment. In some implementations, said first side of said body portion does not comprise an opening. In some implementations, said body portion comprises a flexible, stretchy, and/or resilient material configured to allow a size of said opening to be increased to allow the electronic device to be at least partially inserted through said opening and at least partially positioned within said cavity. In some implementations, said body portion further comprises a frame positioned within said cavity configured to position the electronic device. In some implementations, said frame comprises a resilient material. In some implementations, said body portion and said securement portion are seamlessly integrated with one another. In some implementations, said body portion and said securement portion comprise a first layer, a second layer, and a third layer, said second layer disposed in between said first and third layers, wherein said first side of the body portion comprises said first and second layers, and wherein said second side of the body portion comprises said third layer. In some implementations, said first and third layers comprise a fabric material, and wherein said second layer comprises an unbroken loop fabric material.

Disclosed herein is a wearable device configured to cover and position an electronic device configured to measure a physiological parameter of a subject. The wearable device can comprise: a body portion comprising a first side, a second side opposite the first side, a cavity, and an opening in the second side configured to allow the electronic device to be at least partially inserted through said opening and at least partially positioned within said cavity; and a securement portion connected to the body portion and configured to secure said body portion to the subject to prevent the electronic device from slipping or moving along a tissue site of the subject. Said second side of said body portion can be configured to face toward said tissue site of the subject when the wearable device is secured to the subject. Furthermore, the wearable device can cover the electronic device such that the electronic device is not visible when the securement portion secures the body portion to the subject.

In the above wearable device or in other implementations as described herein, one or more of the following features can also be provided. In some implementations, said securement portion comprises a strap, a band, or a garment. In some implementations, said first side of said body portion does not comprise an opening. In some implementations, said body portion comprises a flexible, stretchy, and/or resilient material configured to allow a size of said opening to be increased to allow the electronic device to be at least partially inserted through said opening and at least partially positioned within said cavity. In some implementations, said body portion further comprises a frame positioned within said cavity configured to position the electronic device. In some implementations, said frame comprises a resilient material. In some implementations, said body portion and said securement portion are seamlessly integrated with one another. In some implementations, said body portion and said securement portion comprise a first layer, a second layer, and a third layer, said second layer disposed in between said first and third layers, wherein said first side of the body portion comprises said first and second layers, and wherein said second side of the body portion comprises said third layer. In some implementations, said first and third layers comprise a fabric material, and wherein said second layer comprises an unbroken loop fabric material.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIG. 1 illustrates a perspective view of a wearable system secured to a subject's wrist in accordance with aspects of this disclosure.

FIGS. 2A-2B illustrate perspective views of the wearable system of FIG. 1 in accordance with aspects of this disclosure.

FIG. 2C illustrates a side view of the wearable system of FIG. 1 in accordance with aspects of this disclosure.

FIGS. 2D-2E illustrate perspective views of the wearable system of FIG. 1 with an electronic device removed from a wearable device in accordance with aspects of this disclosure.

FIG. 2F illustrates a schematic diagram of certain features of an electronic device of the wearable system of FIG. 1 in accordance with aspects of this disclosure.

FIGS. 3A-3D illustrate various views of an implementation of an electronic device in accordance with aspects of this disclosure.

FIGS. 4A-4D illustrate various views of another implementation of an electronic device in accordance with aspects of this disclosure.

FIGS. 5A-5C illustrate various views of a charger of an electronic device in accordance with aspects of this disclosure.

FIGS. 6A-6F illustrate various views of a wearable device of the wearable system of FIG. 1 in accordance with aspects of this disclosure.

FIGS. 7A-7B illustrate perspective views of another implementation of a wearable system in accordance with aspects of this disclosure.

FIG. 7C illustrates a perspective view of the wearable system of FIGS. 7A-7B with an electronic device removed from a wearable device in accordance with aspects of this disclosure

FIGS. 8A-8E illustrate various views of a wearable device of the wearable system of FIGS. 7A-7B in accordance with aspects of this disclosure.

FIGS. 9A-9B illustrate perspective views of another implementation of a wearable system in accordance with aspects of this disclosure.

FIG. 9C illustrates a perspective view of the wearable system of FIGS. 9A-9B with an electronic device removed from a wearable device in accordance with aspects of this disclosure

FIGS. 10A-10F illustrate various views of a wearable device of the wearable system of FIGS. 9A-9B in accordance with aspects of this disclosure.

FIGS. 11A-11B illustrate perspective views of another implementation of a wearable system in accordance with aspects of this disclosure.

FIG. 11C illustrates a perspective view of the wearable system of FIGS. 11A-11B with an electronic device removed from a wearable device in accordance with aspects of this disclosure

FIGS. 12A-12E illustrate various views of a wearable device of the wearable system of FIGS. 11A-11B in accordance with aspects of this disclosure.

FIGS. 13A-13D illustrate various views of another implementation of an electronic device in accordance with aspects of this disclosure.

FIGS. 14A-14B illustrate perspective views of another implementation of a wearable system in accordance with aspects of this disclosure.

FIG. 14C illustrates a perspective view of the wearable system of FIGS. 14A-14B with an electronic device removed from a wearable device in accordance with aspects of this disclosure

FIGS. 15A-15E illustrate various views of a wearable device of the wearable system of FIGS. 14A-14B in accordance with aspects of this disclosure.

FIGS. 16A-16B illustrate perspective views of another implementation of a wearable system in accordance with aspects of this disclosure.

FIG. 16C illustrates a perspective view of the wearable system of FIGS. 16A-16B with an electronic device removed from a wearable device in accordance with aspects of this disclosure

FIGS. 17A-17E illustrate various views of a wearable device of the wearable system of FIGS. 16A-16B in accordance with aspects of this disclosure.

FIGS. 18A-18B illustrate various views of another implementation of a wearable system in accordance with aspects of this disclosure.

FIGS. 19A-19C illustrate various views of a wearable device of the wearable system of FIGS. 18A-18B in accordance with aspects of this disclosure.

FIGS. 20A-20B illustrate various views of another implementation of a wearable system in accordance with aspects of this disclosure.

FIGS. 21A-21C illustrate various views of a wearable device of the wearable system of FIGS. 20A-20B in accordance with aspects of this disclosure.

FIGS. 22A-22E illustrate various views of another implementation of a wearable system in accordance with aspects of this disclosure.

FIGS. 23A-23B illustrate perspective views of another implementation of a wearable system in accordance with aspects of this disclosure.

FIGS. 24A-24C illustrate various views of another implementation of a wearable system in accordance with aspects of this disclosure.

FIGS. 25A-25B illustrate various views of another implementation of a wearable system in accordance with aspects of this disclosure.

FIGS. 26A-26D illustrate various views of another implementation of a wearable system in accordance with aspects of this disclosure.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes or which is essential to practicing the systems, devices, and/or methods disclosed herein.

Disclosed herein are wearable systems that can be used to measure, monitor, transmit (for example, wirelessly or via wired connection), process, and/or determine one or more physiological parameters of a subject (which can also be referred to herein as a “user”, “patient”, or “wearer”). The disclosed wearable systems can generate one or more signals associated with and/or indicative of one or more physiological parameters of a subject and process such one or more signals to determine such physiological parameters. In some implementations, the disclosed wearable systems can generate and transmit one or more signals associated with and/or indicative of one or more physiological parameters of a subject to a separate monitoring, computing, and/or electrical device (for example, wirelessly or via wired connection), for example, a mobile phone, which is capable of processing and/or determining such physiological parameters based on the transmitted signals. Any of the disclosed wearable systems and/or devices in communication with the wearable systems can include hardware and/or software capable of determining and/or monitoring a variety of physiological parameters, including but not limited to blood oxygenation levels in veins and/or arteries, heart rate, blood flow, respiratory rates, an electrocardiogram (ECG) and/or other physiological parameters or characteristics such as those discussed herein. Any of the wearable systems described herein can include and/or employ pulse oximetry (for example, via an optical sensor) to measure physiological parameters of the subject and/or to generate, transmit, and/or process one or more signals associated with and/or indicative of such physiological parameters and/or to determine such physiological parameters. As discussed below, such optical sensor can include one or more emitters configured to emit optical radiation (e.g., light) of one or more wavelengths (e.g., wavelength(s) in the visible spectrum, near infrared wavelength(s), infrared wavelength(s), far infrared wavelength(s), etc.) and one or more detectors configured to detect at least a portion of the emitted optical radiation after attenuation, reflecting off of, and/or passing through tissue of the subject.

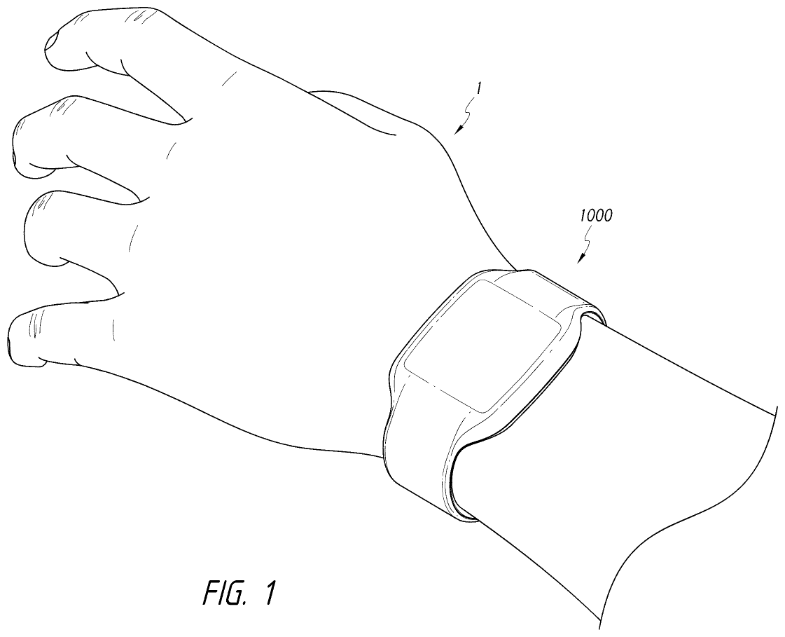

FIG. 1 illustrates a perspective view of a wearable system 1000 (which can also be referred to herein as a “wearable sensor system” or “wearable physiological sensor system”) secured to a wrist of a subject 1. The wearable system 1000 can include an electronic device and a wearable device which are discussed further below. The electronic device can measure one or more physiological parameters of the subject. The wearable device can operably position the electronic device. The wearable device can include a body portion and a securement portion. The body portion of the wearable device can have a cavity for positioning the electronic device, and the securement portion of the wearable device can be connected to the body portion and secure the body portion to the subject 1 as shown and discussed further below.

Although FIG. 1 shows the wearable system 1000 secured to the wrist of the subject 1 in a particular manner which can provide certain advantages as described herein, such illustrated manner and/or location of securement is not intended to be limiting. Wearable system 1000 and various implementations of the wearable systems described herein can be secured to various portions of the subject's body including a wrist, lower arm, upper arm, and/or upper body in a variety of manners and/or using a variety of methods. Alternatively, or in addition, in some implementations the wearable system 1000 and various implementations of the wearable systems described herein can be secured and/or placed adjacent to and/or around portions of a body of the subject 1 other than the wrist, lower arm, upper arm, and/or upper body such as adjacent to and/or around an ankle, a lower leg, an upper leg, a lower body, and/or a torso or mid portion of the subject's body among other regions or portions of the subject's body. Accordingly, while wearable system 1000 and various implementations of the wearable systems disclosed herein may be illustrated and/or described with reference to a wrist of the subject 1, such description is not intended to be limiting, and the wearable system 1000 and various implementations of the wearable systems disclosed herein can be utilized in connection with other portions of the subject's body.

The wearable systems described herein (e.g., wearable systems 1000, 2000, 3000, 4000, 5000, 6000, 7000, 8000, 9000, 10000, 11000, 12000, 13000) and/or aspects thereof, such as the electronic devices described herein (e.g., electronic devices 1200, 1300, 1400, 1500, 1600) and/or the wearable devices described herein (e.g., wearable devices 1100, 2100, 3100, 4100, 5100, 6100, 7100, 8100, 9100, 10100, 11100, 12100, 13100) can be similar or identical to and/or incorporate any of the features described and/or illustrated with respect to any of the devices, assemblies, and/or systems described and/or illustrated in U.S. Pat. No. 11,272,839, filed Oct. 10, 2019, titled “SYSTEM FOR TRANSMISSION OF SENSOR DATA USING DUAL COMMUNICATION PROTOCOL,” in U.S. Pat. No. 9,277,880, filed Jul. 1, 2010, titled “MULTI-STREAM DATA COLLECTION SYSTEM FOR NONINVASIVE MEASUREMENT OF BLOOD CONSTITUENTS,” in U.S. Pat Pub. No. 2021/0290120, filed Jan. 13, 2021, titled “WEARABLE DEVICE WITH PHYSIOLOGICAL PARAMETERS MONITORING,” in U.S. Pat. Pub. No. 2023/0028745, filed Jul. 12, 2022, titled “WEARABLE DEVICE WITH PHYSIOLOGICAL PARAMETERS MONITORING,” and in U.S. Provisional Application No. 63/386,474, filed Dec. 7, 2022, titled “WEARABLE DEVICE WITH PHYSIOLOGICAL PARAMETERS MONITORING,” which are each hereby incorporated by reference in their entirety and for all purposes.

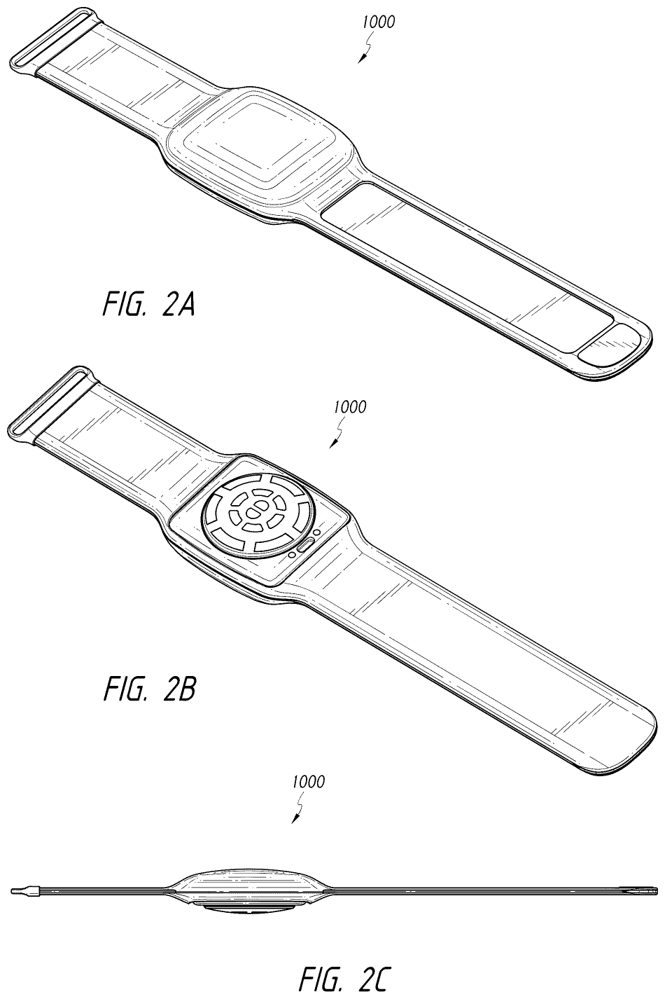

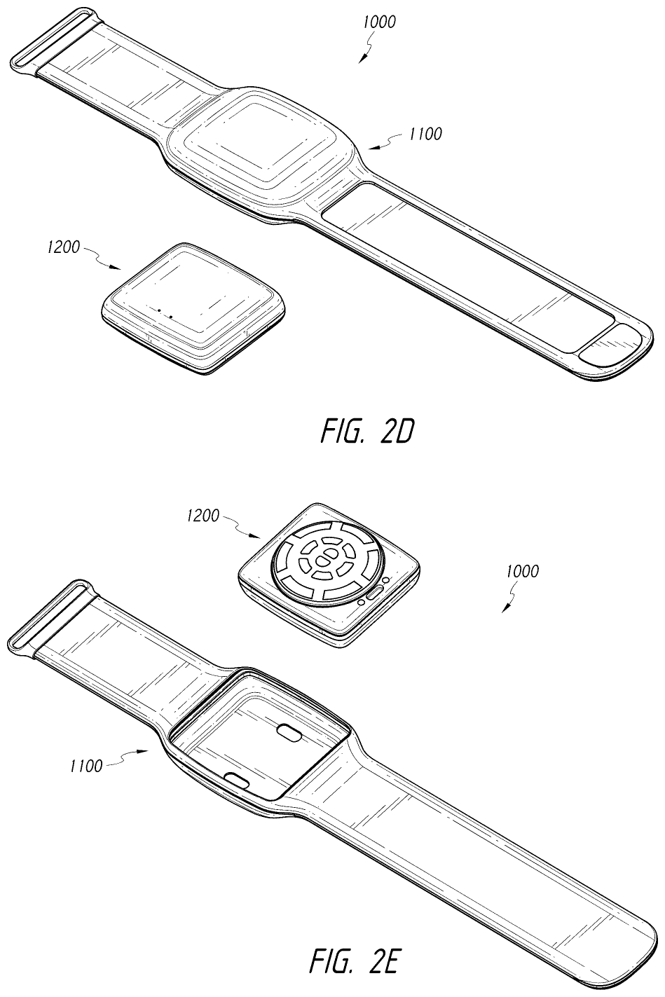

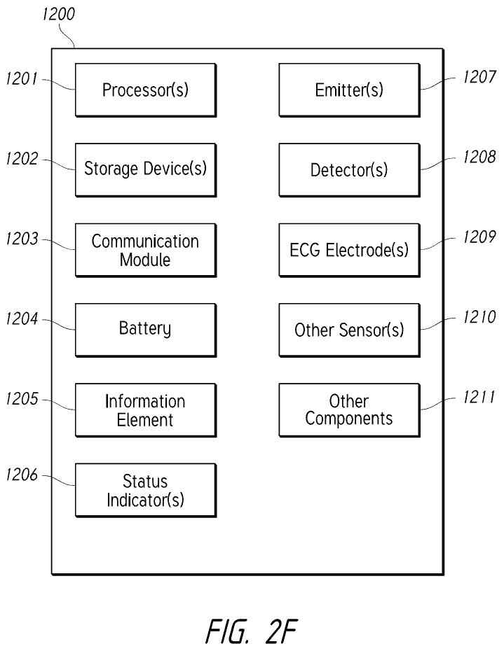

FIGS. 2A-2E illustrate various views of the wearable system 1000 of FIG. 1. The wearable system 1000 can include a wearable device 1100. The wearable device 1100 can be configured to receive, position, and/or cover an electronic device including one or more sensors for measuring one or more physiological parameters of the subject 1. For example, the wearable device 1100 can be configured to receive, position, and/or cover an electronic device 1200 (which can also be referred to herein as an “electronic module,” a “module,” or a “physiological sensor”) or a portion thereof, as described further herein. In some implementations, the wearable system 1000 can include the wearable device 1100 and the electronic device 1200. As shown in FIGS. 2A-2C, the wearable device 1100 and the electronic device 1200 can form a unitary structure configured to be secured to the subject 1 (e.g., a wrist of the subject). FIGS. 2D-2E illustrate the electronic device 1200 disconnected from the wearable device 1100. Although the figures illustrate implementations of the wearable system 1000 in which the wearable device 1100 and the electronic device 1200 are removably connectable to one another, these components may be integrally formed with one another. For example, in some variants, the wearable device 1100 and electronic device 1200 are integrally formed. Implementations of the wearable system 1000 in which electronic device 1200 is removably connectable from wearable device 1100 can advantageously allow for a wearable device 1100 of various sizes (e.g., small, medium, large, extra-large) and/or shapes to be utilized with the wearable system 1000, for example, so as to accommodate various sizes and/or shapes of a subject's body parts (e.g., wrist). In this way, the wearable system 1000 can be customized to a subject 1 by selecting an appropriately configured wearable device 1100 while allowing for other aspects of the wearable system 1000, such as the electronic device 1200, to remain the same and/or be universal across subjects.

FIG. 2F illustrates a schematic diagram of certain features which can be incorporated in wearable system 1000, for example, in the electronic device 1200 of wearable system 1000, as well as any other implementations of the wearable systems and/or associated electronic devices described herein. As shown, the wearable system 1000 (e.g., electronic device 1200 of wearable system 1000) can include one or more emitters 1207 and one or more detectors 1208. Also shown, the wearable system 1000 (e.g., electronic device 1200 of wearable system 1000) can include one or more processors 1201, one or more storage devices 1202, a communication module 1203, a battery 1204, an information element 1205, one or more status indicators 1206, one or more ECG electrodes 1209, one or more other sensors 1210, and/or one or more other components 1211. While certain features of the wearable system 1000 (e.g., the electronic device 1200) are shown in FIG. 2F that can be incorporated therein, any of such features are optional. Furthermore, the wearable system 1000 (e.g., the electronic device 1200) can include other features than shown in FIG. 2F.

The one or more emitters 1207 and the one or more detectors 1208 of the wearable system 1000 (e.g., of the electronic device 1200) can be utilized to obtain physiological information indicative of one or more physiological parameters of the subject. These parameters can include various blood analytes such as oxygen, carbon monoxide, methemoglobin, total hemoglobin, glucose, proteins, glucose, lipids, a percentage thereof (e.g., concentration or saturation), and the like. The one or more emitters 1207 and the one or more detectors 1208 can also be used to obtain a photoplethysmograph, a measure of plethysmograph variability, pulse rate, a measure of blood perfusion, and the like. Information such as oxygen saturation (SpO₂), pulse rate, a plethysmograph waveform, respiratory effort index (REI), acoustic respiration rate (RRa), EEG, ECG, pulse arrival time (PAT), perfusion index (PI), pleth variability index (PVI), methemoglobin (MetHb), carboxyhemoglobin (CoHb), total hemoglobin (tHb), and/or glucose, can be obtained from wearable system 1000 (e.g., from the electronic device 1200) and data related to such information can be processed and/or transmitted by the wearable system 1000 (e.g., via communication module 1203 of the wearable system 1000/electronic device 1200) to a separate device (e.g. a separate computing device such as a mobile phone). The one or more emitters 1207 and the one or more detectors 1208 can be optically based and, for example, utilize optical radiation. Further, the one or more emitters 1207 can serve as a source of optical radiation that can be directed towards tissue (which can also be referred to as a “tissue site”) of the subject 1 when the wearable system 1000 is in use. The wearable system 1000 (e.g., the electronic device 1200) can include one, two, three, four, five, six, seven, or eight or more emitters 1207 and/or one, two, three, four, five, six, seven, or eight or more detectors 1208. The one or more emitters 1207 can be one or more light-emitting diodes (LEDs) (e.g., such as low-power, high-brightness LEDs), laser diodes, incandescent bulbs with appropriate frequency-selective filters, and/or any other source(s) of optical radiation and/or any combinations of the same, or the like. The one or more emitters 1207 can emit optical radiation of one or more wavelengths and can emit visible and near-infrared optical radiation. The one or more emitters 1207 can be similar or identical to any of the emitters discussed in U.S. Pat. Nos. 11,272,839, 9,277,880, U.S. Pat Pub. No. 2021/0290120, U.S. Pat. Pub. No. 2023/0028745, and U.S. Provisional Application No. 63/386,474 incorporated by reference herein. The one or more detectors 1208 can be configured to detect optical radiation generated by the one or more emitters 1208. The one or more detectors 1208 can detect optical radiation that attenuates through and/or is reflected by tissue of the subject 1, for example, tissue of the subject's wrist, lower arm, and/or upper arm. The one or more detectors 1208 can output one or more signals responsive to the detected optical radiation. In some implementations, the one or more detectors 1208 can be one or more photodiodes, phototransistors, or the like. The one or more detectors 1208 can be similar or identical to any of the detectors discussed in U.S. Pat. Nos. 11,272,839, 9,277,880, U.S. Pat Pub. No. 2021/0290120, U.S. Pat. Pub. No. 2023/0028745, and U.S. Provisional Application No. 63/386,474 incorporated by reference herein. Any one or more of the subject's physiological measurements made via the one or more emitters 1207 and the one or more detectors 1208 can be transmitted to a separate device in communication with the wearable system 1000 (e.g., of the electronic device 1200) for display.

The one or more processors 1201 (which can also be referred to herein as “hardware processors) can be configured, among other things, to process data, execute instructions to perform one or more functions, and/or control the operation of the wearable system 1000. For example, the one or more processors 1201 can control operation of the one or more emitters 1207, the one or more detectors 1208, the one or more other sensors 1210, and/or the one or more other components 1211 of the wearable system 1000. As another example, the one or more processors 1201 can process signals and/or physiological data received and/or obtained from the one or more detectors 1208, the one or more other sensors 1210, and/or the one or more other components 1211 of the wearable system 1000. Further, the one or more processors 1201 can execute instructions to perform functions related to storing and/or transmitting such signals and/or physiological data received and/or obtained from the one or more detectors 1208, the one or more other sensors 1210, and/or the one or more other components 1211 of the wearable system 1000. The processor 1201 can execute instructions to perform functions related to storing and/or transmitting any or all of such received data.

The one or more storage devices 1202 can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed physiological data obtained from the wearable system 1000, for example.

The communication module 1203 can facilitate communication (via wires and/or wireless connection) between the wearable system 1000 (and/or components thereof) and separate devices, such as separate monitoring, computing, electrical, and/or mobile devices. For example, the communication module 1203 can be configured to allow the wearable system 1000 to wirelessly communicate with other devices, systems, and/or networks over any of a variety of communication protocols. The communication module 1203 can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module 1203 can allow data and/or instructions to be transmitted and/or received to and/or from the wearable system 1000 and separate computing devices. The communication module 1203 can be configured to transmit (for example, wirelessly) processed and/or unprocessed physiological parameters, data and/or other information to one or more separate computing devices, which can include, among others, a patient monitor, a mobile device (for example, an iOS or Android enabled smartphone, tablet, laptop), a desktop computer, a server or other computing or processing device for display and/or further processing, among other things. Such separate computing devices can be configured to store and/or further process the received physiological parameters, data, and/or other information, to display information indicative of or derived from the received parameters, data, and/or information, and/or to transmit information—including displays, alarms, alerts, and notifications—to various other types of computing devices and/or systems that can be associated with the subject, a hospital, a caregiver (for example, a primary care provider), and/or a user (for example, an employer, a school, friends, family) that have permission to access the subject's data. As another example, the communication module 1203 of the wearable system 1000 can be configured to wirelessly transmit processed and/or unprocessed obtained physiological parameters, data, information and/or other information (for example, motion and/or location data) to a mobile phone which can include one or more hardware processors configured to execute an application that generates a graphical user interface displaying information representative of the processed or unprocessed physiological parameters, data, information and/or other information obtained from the wearable system 1000. The communication module 1203 can be and/or include a wireless transceiver.

The battery 1204 can provide power for hardware components of the wearable system 1000 described herein. The battery 1204 can be rechargeable. For example, the battery 1204 can be a lithium, a lithium polymer, a lithium-ion, a lithium-ion polymer, a lead-acid, a nickel-cadmium, or a nickel-metal hydride battery. In some implementations, the battery 1204 can be charged/recharged by wirelessly charging (e.g., via a wireless charging pad), by solar energy (e.g., via a solar collector if incorporated in the electronic device 1200), and/or by kinetic motion (e.g., via an internal mechanism if incorporated that can convert kinetic motion into electrical power). In some cases, the battery 1204 can be removed, or the battery 1204 can be integrated within and/or a permanent part of the electronic device 1200. In some implementations, the battery 1204 can be non-rechargeable. Additionally or alternatively, the wearable system 1000 can be configured to obtain power from a power source that is external to the wearable system 1000. For example, the wearable system 1000 (e.g., the electronic device 1200) can include or can be configured to connect to a cable which can itself connect to an external power source to provide power to the wearable system 1000.

The information element 1205 can be a memory storage element that stores, in non-volatile memory, information used to help maintain a standard of quality associated with the wearable system 1000. Illustratively, the information element 1205 can store information regarding whether the wearable system 1000 has been previously activated and whether the wearable system 1000 has been previously operational for a prolonged period of time, such as, for example, four hours, one day, two days, five days, ten days, twenty days, a month, multiple months, or any period of time. The information stored in the information element 1205 can be used to help detect improper re-use of the wearable system 1000, for example.

The one or more status indicators 1206 can be configured to provide and/or indicate a status of the wearable system 1000 and/or a status of one or more physiological parameters of the subject 1 determined by the wearable system 1000 and/or any devices in communication with the wearable system 1000. In some implementations, the one or more status indicators 1206 can be configured to indicate a status of the wearable system 1000, such as whether the wearable system 1000 is in an operational (“on”) mode, whether the wearable system 1000 is pairing or has paired with a separate device, whether an error has been detected, and/or a power level of the wearable system 1000 (for example, a charge of battery 1204). For example, the one or more status indicators 1206 can be configured to light up and/or cast optical radiation of one or more wavelengths from one or more portions of the wearable system 1000. As another example, the one or more status indicators 1206 can be configured to light up and/or emit optical radiation from one or more portions of the electronic device 1200 of the wearable system 1000. The one or more processors 1201 can be in communication with the one or more status indicators 1206 and can be configured to instruct the one or more status indicators 1206 to cause any of such above-described status indications and/or lighting. In some cases, the one or more status indicators 1206 can be configured to provide optical radiation (e.g., light) feedback to the subject when the wearable system 1000 is secured to the subject and/or when electronic device 1200 and wearable device 1100 are connected together. In some implementations, wearable system 1000 can be configured to cause optical radiation feedback to the subject 1 (when the wearable system 1000 is secured to the subject) responsive to one or more physiological parameters determined by wearable system 1000 and/or by any devices (such as separate computing and/or mobile devices, for example, a mobile phone) in communication with the wearable system 1000. The one or more processors 1201 can instruct the one or more status indicators 1206 to emit or stop emitting optical radiation and/or instruct the one or more status indicators 1206 to alter a characteristic of optical radiation (for example, increase/reduce optical radiation brightness, change optical radiation wavelength and/or color, change a rate of blinking of optical radiation, etc.) responsive to the one or more determined physiological parameters. Such action by the one or more processors 1201 can dynamically track with physiological parameter determination over time, for example. As an example, in some implementations, the one or more processors 1201 can provide instructions to the one or more status indicators 1206 (such as those discussed above) responsive to a condition of the subject using the wearable system 1000. For example, if one or more physiological parameters determined by the wearable system 1000 and/or any devices in communication with the wearable system 1000 are indicative of hypoxemia (low blood oxygen) when the subject is using the wearable system 1000, the one or more processors 1201 can instruct the one or more status indicators 1206 to produce optical radiation to notify the subject and/or their care providers to restore proper breathing and/or safe blood oxygen levels. As another example, if one or more physiological parameters determined by the wearable system 1000 and/or any devices in communication with the wearable system 1000 are indicative of edema (swelling caused by excess fluid trapped in body tissue) when the subject is using the wearable system 1000, the one or more processors 1201 can instruct the one or more status indicators 1206 to cause optical radiation to be emitted from the wearable system 1000 as described above. In some implementations, the one or more processors 1201 and/or any devices in communication with the wearable system 1000 can instruct the one or more status indicators 1206 to cause optical radiation to be emitted if a determined subject physiological parameter of interest meets and/or exceeds a set threshold, meets and/or falls below a set threshold, and/or meets, exceeds, and/or falls below a set range. In some cases, optical radiation emitted from the one or more status indicators 1206 can correspond to an alert, an alarm, a notification, and/or any other situation wherein the subject and/or a care provider need to intervene in the subject's care. The one or more status indicators 1206 can be positioned within various portions of the wearable system 1000, for example, within electronic device 1200, such that optical radiation emitted from the one or more status indicators emit out of and/or through one or more holes and/or one or more openings in the electronic device 1200, such as shown herein.

The one or more ECG electrodes 1209 of the wearable system 1000 (e.g., of the electronic device 1200) can be utilized to obtain physiological information indicative of one or more physiological parameters of the subject. For example, the one or more ECG electrodes 1209 can be configured to contact the subject (e.g., contact the subject's skin) and output one or more signals responsive to the user's cardiac electrical activity. The one or more processors 1201 can be configured to receive the one or more signals from the ECG electrodes 1209 responsive to the subject's cardiac electrical activity and determine an ECG of the subject responsive to such one or more signals (e.g., automatically determine an ECG of the subject). The wearable system 1000 (e.g., the electronic device 1200) can include one, two, three, four, five, six, seven, eight, nine, ten, or more ECG electrodes 1209. The one or more ECG electrodes 1209 can include one or more negative electrodes, one or more positive electrodes, and one or more reference electrodes. Such negative electrode(s), positive electrode(s), and reference electrode(s) can be electrically isolated from one another. The one or more ECG electrodes 1209 can form an ECG sensor of the wearable system 1000 (e.g., of the electronic device 1200). In some implementations, the wearable system 1000 (e.g., the electronic device 1200) further includes an ECG amplifier configured to receive analog signals from the ECG electrodes 1209, which can output amplified analog signals to an analog-digital converter that can also be included in the wearable system 1000 (e.g., of the electronic device 1200). The amplified analog signals can include an ECG differential between the positive and negative electrodes. The analog-digital converter can output a digital signal based on the analog signals from the one or more ECG electrodes 1209 to the one or more processor 1201 of the wearable system 1000 (e.g., of the electronic device 1200) for determination of the subject's ECG. In some implementations, the one or more ECG electrodes 1209 can optionally make physiological measurements based on the obtained ECG, for example, a heart rate, a respiratory rate, and/or otherwise of the subject. The subject's ECG waveform and/or the other physiological measurements made from the one or more ECG electrodes 1209 can be transmitted to a separate device in communication with the wearable system 1000 (e.g., of the electronic device 1200) for display. The one or more ECG electrodes 1209 can be similar or identical to any of the ECG electrodes discussed in U.S. Pat Pub. No. 2021/0290120, U.S. Pat. Pub. No. 2023/0028745, and U.S. Provisional Application No. 63/386,474 incorporated by reference herein.

In some implementations, the wearable system 1000 can include one or more other sensor(s) 1210. The other sensor(s) 1210 can include one or more of a temperature sensor, a blood pressure monitor, an acoustic sensor (e.g., an audio transducer), a location sensor (e.g., a GPS sensor), and/or any sensor configured to obtain physiological information indicative of one or more physiological parameters of the subject and/or other information of the subject (e.g., a number of steps taken or a distance traveled by the subject). Additionally, or alternatively, such other sensor(s) 1210 can comprise a motion sensor, for example, including one or more accelerometers and/or gyroscopes, that can be utilized to determine motion of the subject and/or a portion of the subject's body (e.g., wrist, lower arm, upper arm, upper body). In some implementations where the wearable system 1000 (e.g., electronic device 1200) includes a motion sensor, the processor(s) 1201 can determine whether the portion of the subject's body that the wearable system 1000 is secured to is moving. Furthermore, in some implementations, the processor(s) 1201 can determine the type of movement being performed and/or an exertion level of the subject. Such determination can be used for a determination of a caloric expenditure, for example. The other sensor(s) 1210 can be disposed on, within, and/or be operably positioned by any one or more of the aspects of the wearable system 1000. For example, the other sensor(s) 1210 can be disposed on, within, and/or be operably positioned by any one or more of the electronic device 1200 and/or the wearable device 1100. The other sensor(s) 1210 can be operably connected to the one or more processors 1201, which can control operation of the other sensor(s) 1210 and/or process data received from the other sensor(s) 1210.

In some implementations, the wearable system 1000 can include one or more other component(s) 1211. The other component(s) 1211 can include one or more of a speaker, a vibration motor, or any other component. In some implementations, the one or more other components 1211 can be configured to produce an output to the subject. For example, the wearable system 1000 (e.g., the electronic device 1200) can include a vibration motor that can be configured to vibrate one or more portions of the wearable system 1000 (e.g., at least the electronic device 1200), which in turn can vibrate one or more portions of the subject's body when the wearable system 1000 is secured to the subject. The one or more processors 1201 can be in communication with vibration motor and can be configured to instruct the vibration motor to cause any of such above-described vibration. In some cases, the vibration motor can be utilized to provide haptic feedback to the subject when the wearable system 1000 is secured to the subject. In some implementations, the wearable system 1000 can be configured to cause vibration of and/or provide haptic feedback to one or more portions of the subject's body (when the wearable system 1000 is secured to the subject) via the vibration motor responsive to one or more physiological parameters determined by wearable system 1000 and/or by any devices (such as separate computing, electrical, and/or mobile devices, for example, a patient monitor 10) in communication with the wearable system 1000. The one or more processors 1201 can instruct the vibration motor to cause vibration, cease vibrating, and/or instruct the vibration motor to alter a characteristic of vibration (for example, increase/reduce vibration rate, increase/reduce vibration strength, change vibration pattern, etc.) responsive to the one or more determined physiological parameters. Such action by the one or more processors 1201 can dynamically track with physiological parameter determination over time, for example. As an example, in some implementations, the one or more processors 1201 can provide instructions to the vibration motor (such as those discussed above) responsive to a condition of the subject using the wearable system 1000. For example, if one or more physiological parameters determined by the wearable system 1000 and/or any devices in communication with the wearable system 1000 are indicative of hypoxemia (low blood oxygen) when the subject is using the wearable system 1000 (e.g., such as when sleeping), the one or more processors 1201 can instruct the vibration motor to vibrate to cause the subject to wake up in an attempt to restore proper breathing and/or safe blood oxygen levels. As another example, if one or more physiological parameters determined by the wearable system 1000 and/or any devices in communication with the wearable system 1000 are indicative of edema (swelling caused by excess fluid trapped in body tissue) when the subject is using the wearable system 1000, the one or more processors 1201 can instruct the vibration motor to cause vibration of a portion of the subject's body. In some implementations, the one or more processors 1201 and/or any devices in communication with the wearable system 1000 can instruct the vibration motor to cause a vibration if a determined subject physiological parameter of interest meets and/or exceeds a set threshold, meets and/or falls below a set threshold, and/or meets, exceeds, and/or falls below a set range. In some cases, a vibration of the vibration motor can correspond to an alert, an alarm, a notification, and/or any other situation wherein the subject and/or a care provider need to intervene in the subject's care. In some implementations, the one or more processors 1201 can instruct the vibration motor to vibrate responsive to a status of battery 1204 (for example, when a charge of the battery 1204 drops below a certain threshold). In some implementations, wearable system 1000 can include more than one vibration motor, for example, two, or three or more vibration motors. Vibration motor(s) if included can be positioned within various portions of the wearable system 1000, for example, within electronic device 1200.

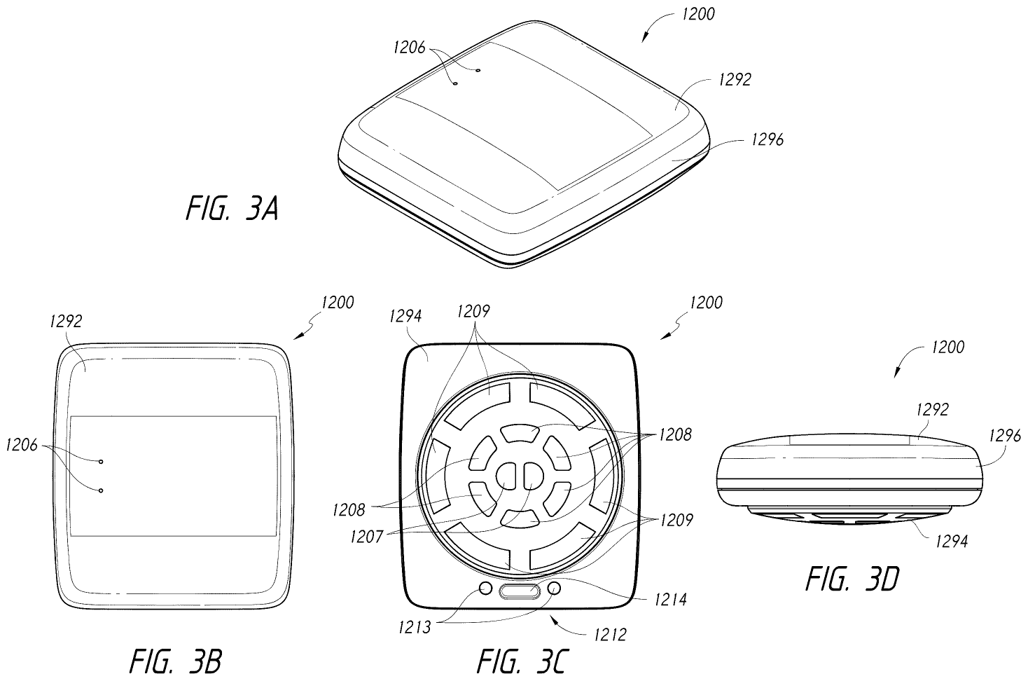

FIG. 3A-3D illustrate various views of the electronic device 1200 that can be incorporated into any of the wearable systems described herein (e.g., in wearable system 1000). FIG. 3A shows a top perspective view, FIG. 3B shows a top view, FIG. 3C shows a bottom view, and FIG. 3D shows a side view of the electronic device 1200. The electronic device 1200 can include any one or more of the features illustrated and discussed with respect to FIG. 2F, including the one or more processors 1201, the one or more storage devices 1202, the communication module 1203, the battery 1204, the information element 1205, the one or more status indicators 1206, the one or more emitters 1207, the one or more detectors 1208, the one or more ECG electrodes 1209, the one or more other sensors 1210, and/or the one or more other components 1211. Furthermore, the electronic device can be similar or identical to and/or incorporate any of the features described and/or illustrated with respect to any of the devices, assemblies, and/or systems described and/or illustrated in U.S. Pat Pub. No. 2021/0290120, U.S. Pat. Pub. No. 2023/0028745, and U.S. Provisional Application No. 63/386,474 incorporated by reference herein.

As shown in the top perspective, top, and bottom views of the electronic device 1200 in FIGS. 3A-3C, respectively, when viewed from above the electronic device 1200 can have a generally rounded rectangular shape. Such a rounded rectangular shape can allow the electronic device 1200 to fit into a wearable device as described herein (e.g., wearable device 1100) in two orientations (each 180 degrees from each other). This can advantageously allow for the electronic device 1200 to be positioned in the wearable device as desired and secured to either a left wrist or a right wrist of a subject among other body locations and sides (e.g., left or right). When viewed from the side, such as shown in FIG. 3D, the electronic device 1200 can have a rounded side 1296 with rounded transitions between the side 1296 and the top 1292 and the side 1296 and the bottom 1294. Additionally, the side 1296 of the electronic device 1200 can taper inwards towards the center of the electronic device 1200 from near the bottom 1294 to the top 1292, which can facilitate securement and removal of the electronic device 1200 with a wearable device as described herein (e.g., wearable device 1100).

As shown in FIGS. 3A-3B, the one or more status indicators 1206 of the electronic device 1200 described with respect to FIG. 2F, when included, can be disposed along the top 1292 of the electronic device 1200. Such positioning can enable a subject to view light emitted from the status indicator(s) 1206 when the wearable system 1000 is in use depending upon the wearable device utilized. As shown in FIG. 3C, the one or more emitters 1207 and the one or more detectors 1208 can be disposed along the bottom 1294 of the electronic device 1200. Additionally, when included, the one or more ECG electrodes 1209 can be disposed along the bottom 1294 of the electronic device 1200. Such positioning of the one or more emitters 1207, the one or more detectors 1208, and the one or more ECG electrodes 1209 can facilitate physiological measurements of the subject when the electronic device 1200 is positioned by a wearable device described herein. For example, the bottom 1294 can be positioned by a wearable device as described herein such that it is placed against a tissue site of the subject (e.g., against skin of the subject). As shown in FIG. 3C, the one or more emitters 1207 can be positioned substantially about the center of the bottom 1294 of the electronic device 1200. Further as shown in FIG. 3C, the one or more detectors 1208 can be positioned around the one or more emitters 1207, for example, in a substantially semi-annular/semi-circular configuration that at least partially surrounds the one or more emitters 1207. Additionally, the one or more ECG electrodes 1209 can be positioned around the one or more emitters 1207 and the one or more detectors 1208, for example, in a substantially semi-annular/semi-circular configuration that at least partially surrounds the one or more emitters 1207 and the one or more detectors 1208.

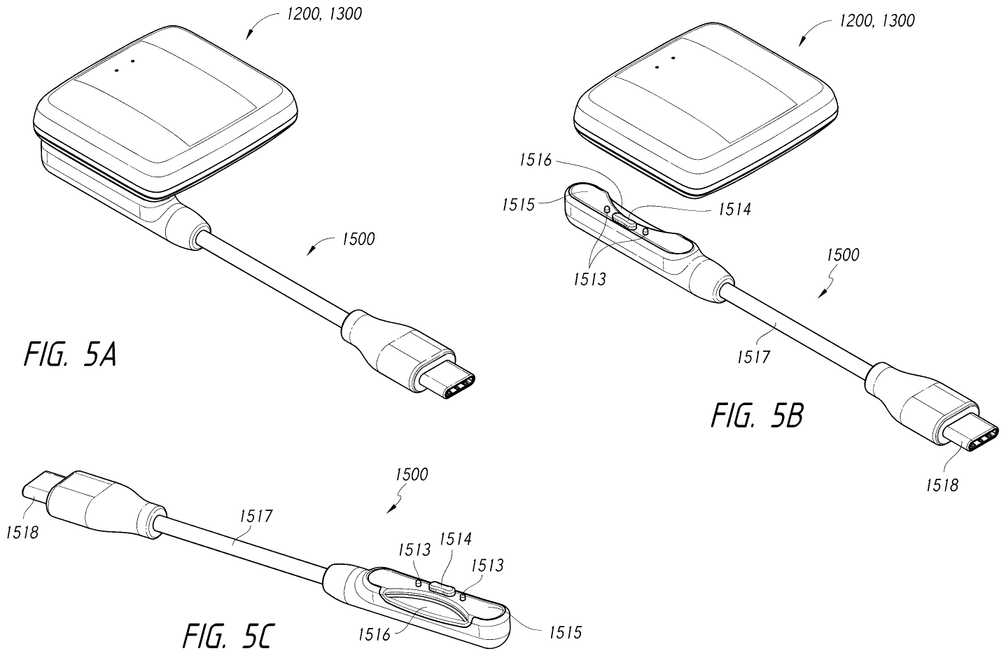

As described with respect to FIG. 2F, the electronic device 1200 can be powered by a battery 1204. The battery 1204 can be rechargeable. The battery 1204 can be charged/recharged by connecting the electronic device 1200 to a source of electrical power. For this, the electronic device 1200 can include a charge port 1212 configured to charge/recharge the battery 1204. Such port 1212 can be disposed on the bottom 1294. The charge port 1212 can include electrical contacts 1213 to electrically connect the battery 1204 to a source of power. The charge port 1212 can also include a feature 1214 configured to aid in positioning corresponding electrical contacts of a charger (e.g., charger 1500 illustrated and described with respect to FIGS. 5A-5C) with the electrical contacts 1213 of the charge port 1212. In some implementations, the feature 1214 can comprise a magnet configured to aid in positioning a charger with the charge port via interaction with a corresponding magnet of the charger (e.g., a magnet of an opposite polarity). Additionally or alternatively, the feature 1214 can include a recess (e.g., an oblong recess as shown) configured to aid in positioning a charger with the charge port via interaction with a corresponding feature of the charger (e.g., an oblong protrusion).

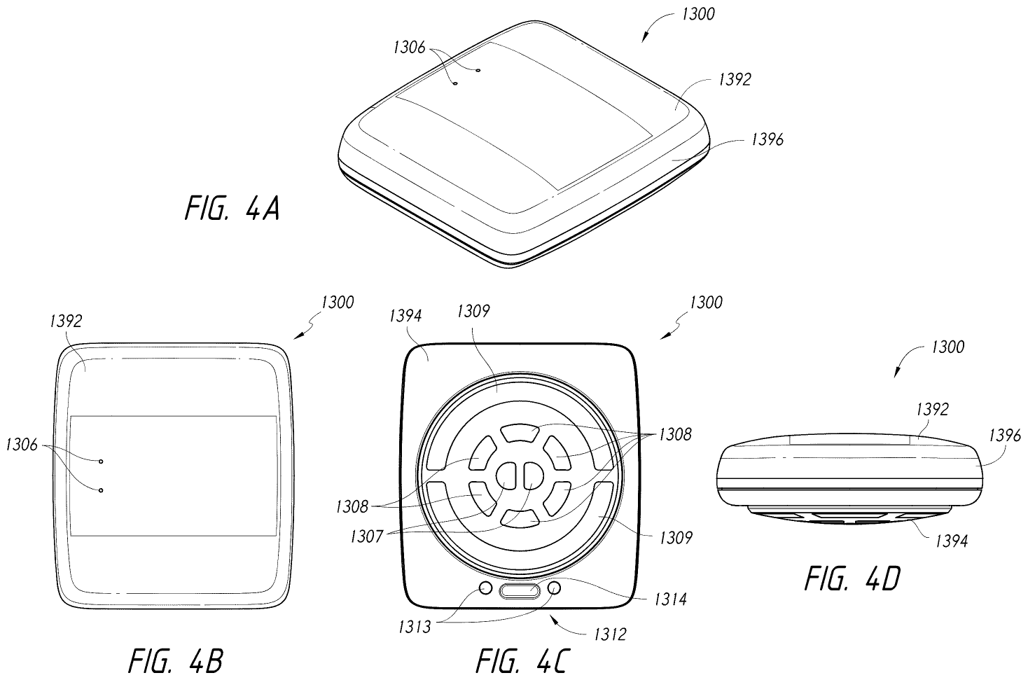

FIG. 4A-4D illustrate various views of an electronic device 1300 that can be incorporated into any of the wearable devices described herein. FIG. 4A shows a top perspective view, FIG. 4B shows a top view, FIG. 4C shows a bottom view, and FIG. 4D shows a side view of the electronic device 1200. The electronic device 1300 is identical to the electronic device 1200 and includes all features and/or functionality of the electronic device 1200 except that its one or more ECG electrodes 1309, when included, can have a different configuration than the one or more ECG electrodes 1209 of the electronic device 1200. For example, the electronic device 1300 can have a top 1392, a bottom 1394, a side 1396, and an overall shape that is identical to the top 1292, the bottom 1294, the side 1296, and the overall shape of the electronic device 1200. Furthermore, the electronic device 1300 can have one or more status indicators 1306, one or more emitters 1307, and one or more detectors 1308 that are identical to the one or more status indicators 1206, the one or more emitters 1207, and the one or more detectors 1208 of the electronic device 1200. The electronic device 1300 can also have a charge port 1312 including electrical contacts 1313 and a feature 1314 that are identical to the charge port 1212 including electrical contacts 1213 and the feature 1214 of the electronic device 1200. Additionally, any of the features of the electronic device 1200 that are described with respect to FIG. 2F can be included in the electronic device 1300. As an example of the different ECG electrode configuration and as shown in FIG. 4C, the one or more ECG electrodes 1308 of electronic device 1300 can include two distinct ECG electrodes in a substantially semi-annular/semi-circular configuration rather than the six shown as an example in FIG. 3C for electronic device 1200.

FIGS. 5A-5C illustrate an implementation of a charger 1500 for charging/recharging the battery of the electronic device 1200, 1300. FIG. 5A shows a perspective view of the charger 1500 operably connected to the electronic device 1200, 1300, FIG. 5B shows a perspective view of the charger 1500 disconnected from the electronic device 1200, 1300, and FIG. 5C shows a perspective view of the charger 1500. As shown, the charger 1500 can include an adapter 1518, a cable 1517, and electrical contacts 1513. Furthermore and as shown, the charger 1500 can include a feature 1514, and/or contours 1515, 1516. The adapter 1518 can be configured to connect to a source of electrical power. In some implementations, the adapter 1518 can include a USB-C type charger. The cable 1517 can mechanically and electrically connect the adapter 1518 to the electrical contacts 1513. The electrical contacts 1513 can be configured to mechanically and electrically connect with the electrical contacts 1213, 1313 of the electronic devices 1200, 1300 described herein. The feature 1514 can be configured to aid in positioning the electrical contacts 1513 with the corresponding electrical contacts 1213, 1313 of the electronic device 1200, 1300. In some implementations, the feature 1514 can comprise a magnet configured to aid in positioning the charger 1500 (e.g., the electrical contacts 1513 thereof) with the charge port 1212, 1213 (e.g., the electrical contacts 1213, 1313 thereof) via interaction with a corresponding magnet of the charge port 1212, 1312 (e.g., a magnet of an opposite polarity). Additionally or alternatively, the feature 1514 can include a protrusion (e.g., an oblong protrusion as shown) configured to aid in positioning the charger 1500 with the charge port 1212, 1312 via interaction with the corresponding feature 1214, 1314 of the charge port 1212, 1312 (e.g., an oblong recess). The contours 1515, 1516 can be configured to aid in positioning the electrical contacts 1513 with the corresponding electrical contacts 1213, 1313 of the electronic device 1200, 1300. For example, the contours 1515, 1516 can include dished contours that complement portions of the bottom 1294, 1394 of the electronic device 1200, 1300.

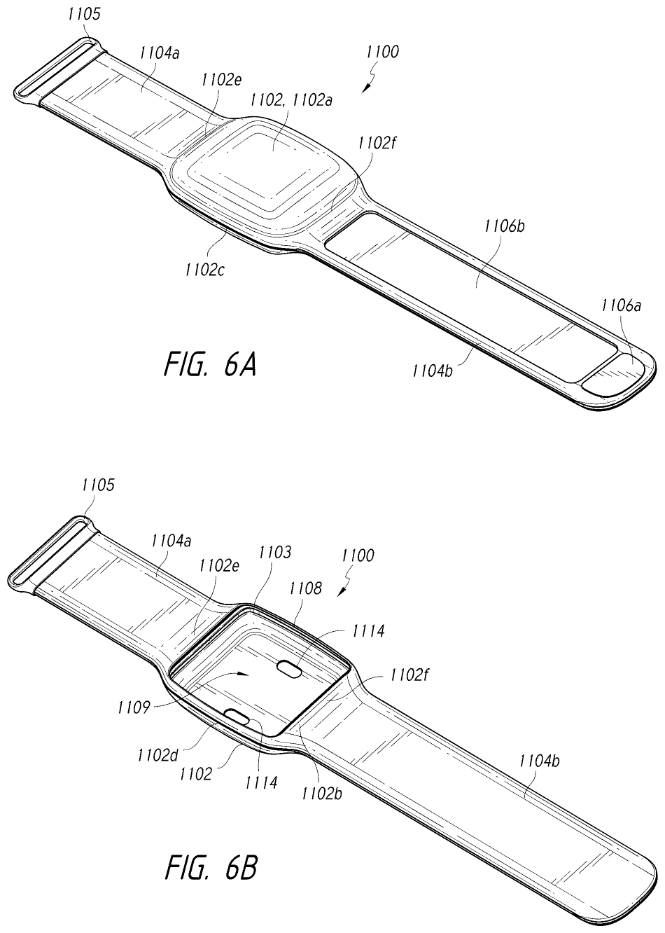

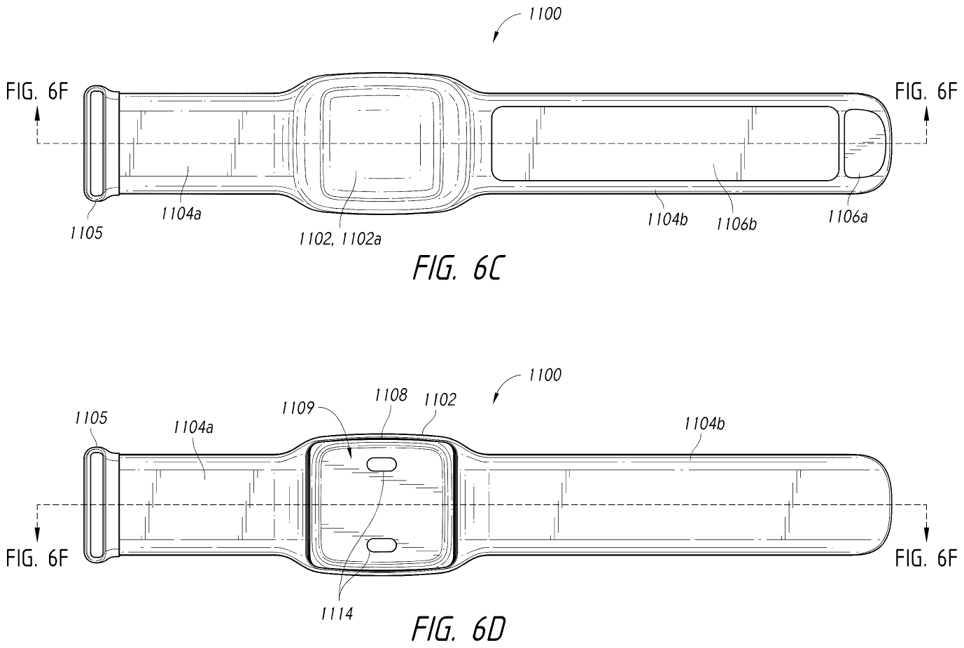

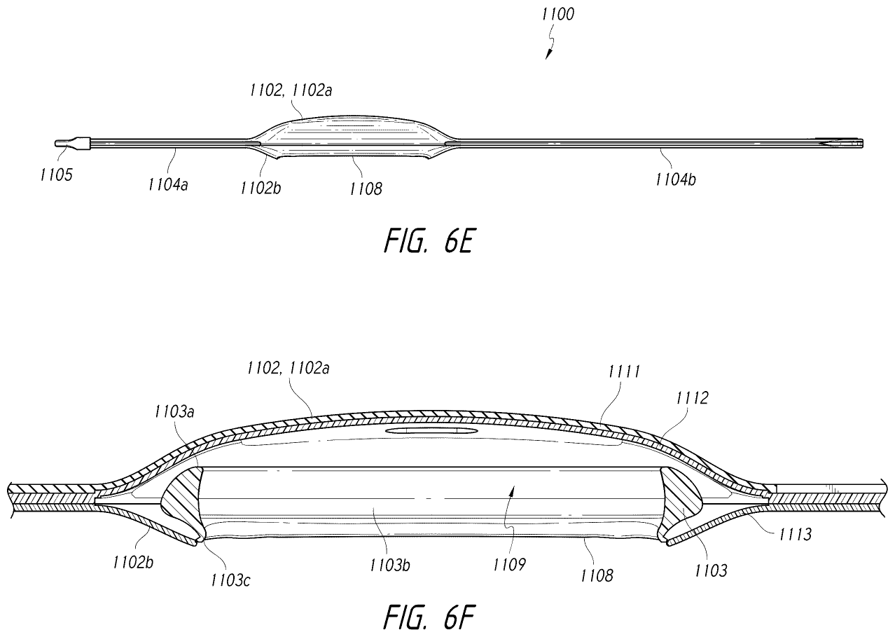

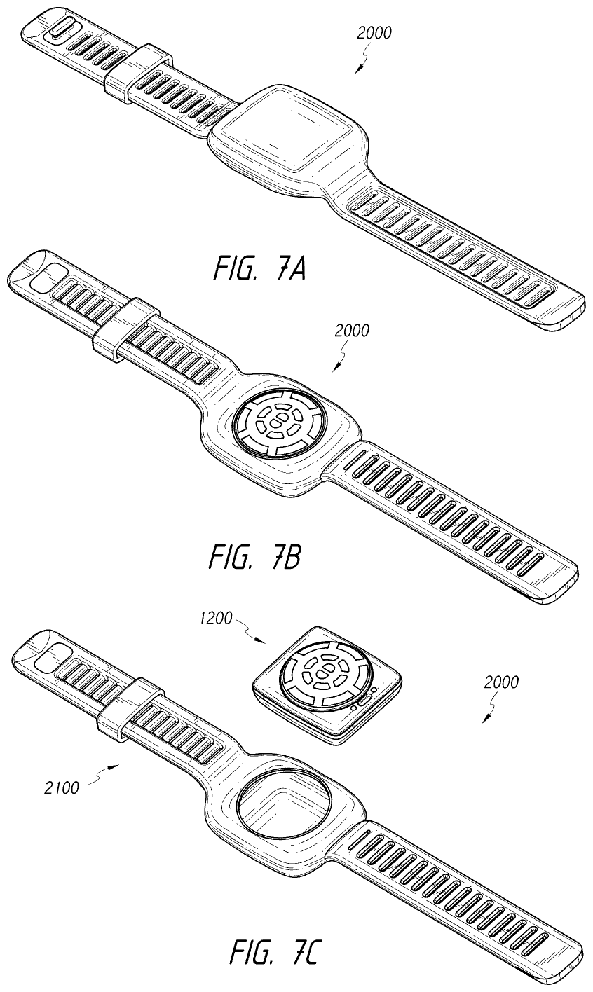

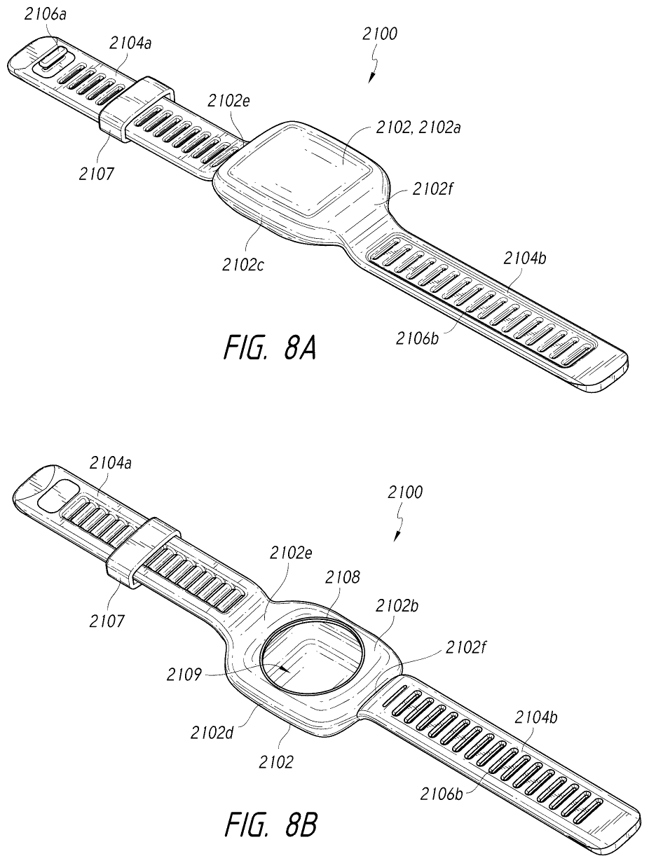

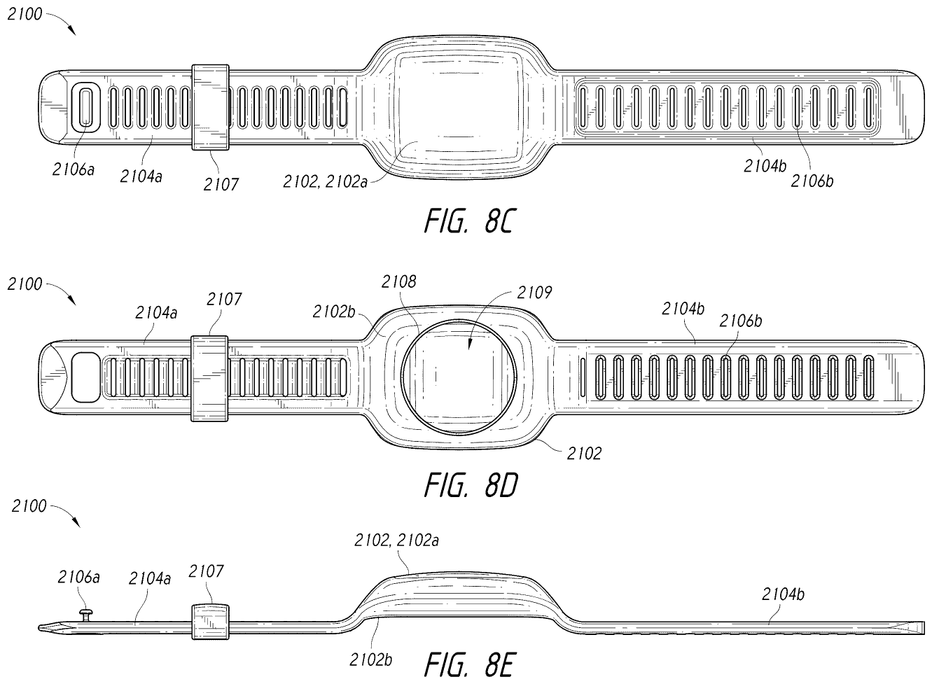

FIGS. 6A-6F illustrate various views of the wearable device 1100 of the wearable system 1000. FIG. 6A illustrates a top perspective view, FIG. 6B illustrates a bottom perspective view, FIG. 6C illustrates a top view, FIG. 6D illustrates a bottom view, FIG. 6E illustrates a side view, and FIG. 6F illustrates a cross-sectional side view of a portion of the wearable device 1100 as indicated in FIGS. 6C-6D. Wearable device 1100 can be configured to secure around a portion of a subject's body (e.g., a wrist as shown in FIG. 1). Wearable device 1100 can include a body portion 1102 and a securement portion connected to the body portion 1102. Such securement portion can include one or both of straps 1104a, 1104b as shown. Such straps 1104a, 1104b can connect to one another via a loop 1105 which can, for example, be disposed at an end of strap 1104a as shown. The straps 1104a, 1104b can be integrally formed with the body portion 1102 (e.g., from the same material). In some implementations, the connection between straps 1104a, 1104b and the body portion 1102 is seamless. In some implementations, strap 1104b can be inserted through loop 1105 and secured to itself, for example, via an attachment mechanism (such as a magnetic attachment and/or a hook-and-loop type attachment, by connecting attachment portions 1106a, 1106b).

Body portion 1102 can have a first side 1102a (which can also be referred to as a “top face” or a “top portion”), a second side 1102b opposite the first side 1102a (and which can also be referred to as a “bottom face” or a “bottom portion”), ends 1102c, 1102d (which can be opposite one another and generally parallel to one another), and ends 1102e, 1102f (which can be opposite one another and generally parallel to one another). The securement portion including straps 1104a, 1104b, can connect to the body portion 1102 at ends 1102e, 1102f, respectively. Body portion 1102 can include a cavity 1109. Body portion 1102 can include an opening 1108 in the second side 1102b. Opening 1108 can be configured to allow the electronic device 1200 (or any of the electronic devices disclosed herein) to be at least partially inserted through the opening 1108 and at least partially positioned within the cavity 1109. For this, the cavity 1109 can be configured to at least partially receive and position the electronic device 1200. The second side 1102b of the body portion 1102 can be configured to face toward a tissue site of the subject (e.g., toward skin of the subject) when the wearable device 1100 is secured to the subject. Such configuration can allow the one or more emitters 1207, the one or more detectors 1208, and/or the one or more ECG electrodes 1209 of the electronic device 1200 to contact and/or face toward the tissue site of the subject (e.g., toward skin of the subject) via the opening 1108 (e.g., by being positioned within and/or through the opening 1108). For example, FIGS. 2B-2C illustrate the electronic device 1200 positioned by the wearable device such that the one or more emitters 1207, the one or more detectors 1208, and/or the one or more ECG electrodes 1209 of the electronic device 1200 are accessible to contact and/or face toward the tissue site of the subject via the opening 1108.

In some implementations, body portion 1102 comprises a flexible, stretchy, and/or resilient material configured to allow a size of the opening 1108 to be increased to allow the electronic device 1200 to be at least partially inserted through the opening 1108 and at least partially positioned within the cavity 1109. Furthermore, in some implementations, body portion 1102 comprises a flexible, stretchy, and/or resilient material configured to allow a size of the cavity 1109 to be increased to allow the electronic device 1200 to be at least partially positioned within the cavity 1109. Such material of the body portion 1102 can advantageously allow the electronic device 1200 to be held securely by the body portion 1102 and aid in preventing the electronic device 1200 from slipping or moving along a tissue site of the subject. In some implementations, the securement portion including straps 1104a, 1104b comprises a flexible, stretchy, and/or resilient material. Such material of the securement portion can advantageously allow the securement portion to secure the body portion 1102 to the subject and prevent the electronic device 1200 held by the body portion 1102 from slipping, moving, and/or coming away from the tissue site of the subject (e.g., the skin of the subject). In some implementations, at least a portion of the wearable device 1100 comprises a fabric material. For example, at least a portion of the body portion 1102 and/or at least a portion of the securement portion including straps 1104a, 1104b can comprise a fabric material.

In some implementations, wearable device 1100 (e.g., the body portion 1102) includes a frame 1103 positioned within cavity 1209 that is configured to removably secure to the electronic device 1200. In some implementations, such frame 1103 comprises a material that is more rigid than a material from which the body portion 1102 and/or the securement portion including straps 1104a, 1104b is made. The frame 1103 can comprise a resilient material that can allow the frame 1103 to deform and/or flex when securing to the electronic device 1200. In some implementations, frame 1103 includes engagement features to retain and/or position the electronic device 1200 within the wearable device 1100 (e.g., at least partially within cavity 1109). As shown in FIG. 6F, the frame 1103 can have an upper ridge 1103a, a lower ridge 1103c, and an inner portion 1103b spanning between the upper ridge 1103a and the lower ridge 1103c. The upper ridge 1103a can be a continuous or a discontinuous ridge along a top portion of the frame 1103 that prevents/limits further upward movement of the electronic device 1200 within the body portion 1102 when the electronic device 1200 and wearable device 1100 are secured to one another. For example, a portion of the top 1292 and/or an upper portion of the side 1296 of the electronic device 1200 can seat against the upper ridge 1103a when the electronic device 1200 and wearable device 1100 are secured to one another. The inner portion 1103b of the frame 1103 can be configured to receive the side 1296 of the electronic device 1200 when the electronic device 1200 and wearable device 1100 are secured to one another. The lower ridge 1103c of the frame 1103 can be a continuous or discontinuous ridge along a bottom portion of the frame 1103 that allows the electronic device 1200 to be removably received by the body portion 1102 and secures the electronic device 1200 within the body portion 1102 when the electronic device 1200 and wearable device 1100 are secured to one another. For example, a portion of the bottom 1294 and/or a lower portion of the side 1296 of the electronic device 1200 can seat against the lower ridge 1103c of the frame 1103 when the electronic device 1200 and wearable device 1100 are secured to one another. In some implementations, the releasable connection between the frame 1103 and the electronic device 1200 is a snap fit.

In some implementations and as shown in FIGS. 1 & 2A, the wearable device 1100 covers the electronic device 1200 such that the electronic device 1200 is not visible when the securement portion secures the body portion 1102 to the subject. For this, a material of at least a portion of the wearable device 1100 can be opaque and/or non transparent (e.g., the body portion 1102 can be opaque and/or non transparent). Furthermore, in some implementations, the first side 1102a of body portion 1102 does not include an opening. Such a lack of an opening in the first side 1102a can, as examples, prevent access to at least a portion of the electronic device 1200 (e.g., the top 1292), further prevent a visibility of the electronic device 1200, and/or provide a smooth, continuous, and/or comfortable cover over at least a portion of the electronic device 1200 when the electronic device 1200 is secured by the wearable device 1100. In some implementations, status indicators 1206 of the electronic device 1200 can be visible through the first side 1102a of the body portion 1102 while a remainder of the electronic device 1200 is not visible. For this, the first side 1102a of the body portion can include one or more recesses 1114 that extend at least partially through a thickness of the first side 1102a and substantially align with status indicators 1206 when the electronic device 1200 is secured by the wearable device 1100. Alternatively, or in addition, the material of the body portion 1102 can allow light from the status indicators 1206 at least partially therethrough while still preventing a visibility of the electronic device 1200.

In some implementations and as shown in FIG. 2B, the wearable device 1100 can be configured to allow the charge port 1212 of the electronic device 1200 to be accessible for charging the battery 1204 of the electronic device 1200 when the electronic device 1200 is at least partially positioned within the cavity 1109. Such configuration can advantageously allow a subject to charge the battery 1204 of the electronic device 1200 without having to remove the electronic device 1200 from the wearable device 1100.

With reference to FIG. 6F, in some implementations the body portion 1102 and/or the securement portion including straps 1104a, 1104b comprise a first layer 1111, a second layer 1112, and a third layer 1113, with the second layer 1112 disposed in between the first layer 1111 and the third layer 111. In such implementations, the first side 1102a of the body portion 1102 can comprise the first layer 1111 and the second layer 1112, and the second side 1102b of the body portion 1102 can comprise the third layer 1113. The opening 1108 can thus be formed in the lower layer 1113. Furthermore, in such implementations, the first layer 1111 and the third layer 1113 can comprise a fabric material, and the second layer 1112 can comprise an unbroken loop fabric material. The strap 1104b can thus have at least a portion of the first layer 1111 removed from a top portion thereof (e.g., as shown in FIGS. 6A & 6C) to expose the second layer 1112 and form attachment portion 1106b comprising such unbroken loop fabric. Furthermore, in implementations in which the wearable device 1100 includes the frame 1103, the frame 1103 can connect to the third layer 1113 of the body portion 1102 (see FIG. 6F). For example, a bottom portion of the frame 1103 adjacent the lower ridge 1103c can connect to the third layer 1113 about the opening 1108. In some implementations in which the body portion 1102 includes one or more recesses 1114, such recesses 1114 can comprise openings in the second layer 1112. In a variant of the wearable device 1100, the wearable device 1100 can comprise two layers rather than three, with the first layer 1111 and the second layer 1112 instead comprising a single layer.

In some implementations, the wearable device 1100 can include features and/or materials to enhance a grip of the wearable device 1100 onto skin of the subject to prevent the electronic device from slipping or moving along the tissue site of the subject. Such features can include bumps, a roughened surface texture, and the like. Such materials can include a tacky and/or rubber-like material (e.g., a silicone and/or silicone rubber), an adhesive material, or the like. In some implementations, such features can comprise such materials. Furthermore, such features and/or materials can be disposed at least partially along a body-contacting side of the wearable device 1100 (e.g., the bottom of the wearable device 1100).

In some implementations and as shown, the body portion 1102 of the wearable device 1100 can have a width (e.g., extending between ends 1102c, 1102d) that is greater than a width of the securement portion (e.g., a width of the straps 1104a, 1104b). In variants of the wearable device 1100, the body portion 1102 can have a width (e.g., extending between ends 1102c, 1102d) that is substantially similar or the same as a width of the securement portion (e.g., a width of the straps 1104a, 1104b).

FIGS. 7A-7C illustrate various views of another implementation of a wearable system 2000. The wearable system 2000 can be similar to the wearable system 1000 and can include any or all features and/or functionality of the wearable system 1000. The wearable system 2000 can include a wearable device 2100. The wearable device 2100 can be configured to receive, position, and/or cover the electronic device 1200 including one or more sensors for measuring one or more physiological parameters of the subject 1 similar or the same as the wearable device 1100 of wearable system 1000. In some implementations, the wearable system 2000 can include the wearable device 2100 and the electronic device 1200. As shown in FIGS. 7A-7B, the wearable device 2100 and the electronic device 1200 can form a unitary structure configured to be secured to the subject 1 (e.g., a wrist of the subject). FIG. 7C illustrates the electronic device 1200 disconnected from the wearable device 2100. Although the figures illustrate implementations of the wearable system 2000 in which the wearable device 2100 and the electronic device 1200 are removably connectable to one another, these components may be integrally formed with one another. For example, in some variants, the wearable device 2100 and electronic device 1200 are integrally formed.

FIGS. 8A-8E illustrate various views of the wearable device 2100 of the wearable system 2000. FIG. 8A illustrates a top perspective view, FIG. 8B illustrates a bottom perspective view, FIG. 8C illustrates a top view, FIG. 8D illustrates a bottom view, and FIG. 8E illustrates a side view of the wearable device 2100. The wearable device 2100 can be similar to the wearable device 1100 and can include any or all features and/or functionality of the wearable device 1100. For example, the wearable device 2100 can include a body portion 2102 and a securement portion connected to the body portion 2102 which can be similar to the body portion 1102 and securement portion connected thereto described with respect to the wearable device 1100. Further to this example, the body portion 2102 can include a first side 2102a, a second side 2102b opposite the first side 2102a, ends 2102c, 2102d, ends 2102e, 2102f, a cavity 2109, and an opening 2108 which can be similar to the body portion 1102, first side 1102a, second side 1102b, ends 1102c, 1102d, ends 1102e, 1102f, cavity 1109, and opening 1108 of the wearable device 1100. The wearable device 2100 can differ from the wearable device 1100 in that it can have a different securement portion configuration as described further below. In some implementations, the wearable device 2100 can also differ from the wearable device 1100 in that it can be a formed (e.g., molded) structure. Such structure can comprise a silicone and/or a silicone rubber. Furthermore, such structure can be without multiple layers. The wearable device 2100 can also differ from the wearable device 1100 in that it can be without a frame similar or identical to the frame 1103 (e.g., the body portion 2102 may not include a frame within cavity 2109). In such implementations, the body portion 2102 can be configured to position and secure the electronic device 1200 without such a frame. In some variants, the wearable device 2100 can include a frame similar or identical to the frame 1103 of wearable device 1100.

The securement portion of the wearable device 2100 can include straps 2104a, 2104b and attachment portions 2106a, 2106b. The securement portion of the wearable device 2100 can also include a strap loop 2107. While the securement portion including straps 2104a, 2104b and attachment portions 2106a, 2106b can function similarly to the securement portion including straps 1104a, 1104b and attachment portions 1106a, 1106b of the wearable device 1100, they can be implemented in a different way. As shown in FIGS. 8A-8E, the straps 2104a, 2104b can be integrally formed with the body portion 2102 (e.g., from the same material). In some implementations, the connection between straps 2104a, 2104b and the body portion 2102 is seamless. Such straps 2104a, 2104b can connect to one another via interaction between the attachment portions 2106a, 2106b. Attachment portion 2106a can include a knobbed protrusion (e.g., an oblong knobbed protrusion as shown) that extends from strap 2104a (e.g., near an end of strap 2104a). Attachment portion 2106b can include one or more openings (e.g., oblong openings) that extend through the strap 2104b (e.g., along at least a portion of a length of strap 2104b). Thus, in some implementations, the straps 2104a, 2104b can connect to one another by the knobbed protrusion of attachment portion 2106a being inserted through one of the openings of attachment portion 2106b. To aid in maintaining such connection of the straps 2104a, 2104b, at least a portion of strap 2104b can be inserted through strap loop 2107 disposed around strap 2104a.

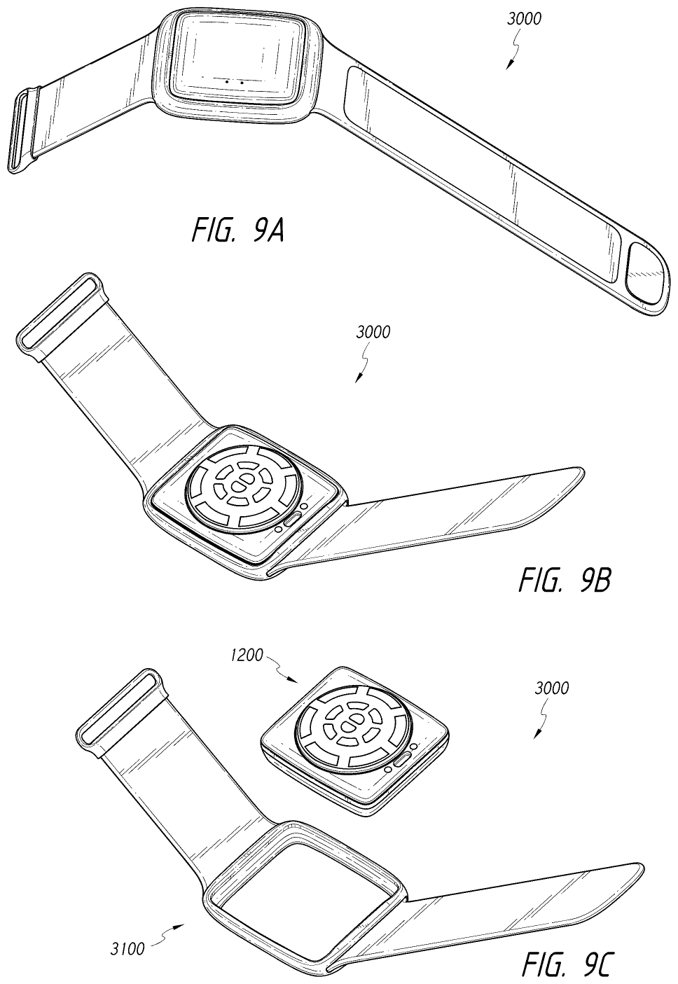

FIGS. 9A-9C illustrate various views of another implementation of a wearable system 3000. The wearable system 3000 can be similar to the wearable system 1000 and can include any or all features and/or functionality of the wearable system 1000. The wearable system 3000 can include a wearable device 3100. The wearable device 3100 can be configured to receive, position, and/or at least partially cover the electronic device 1200 including one or more sensors for measuring one or more physiological parameters of the subject 1 similar or the same as the wearable device 1100 of wearable system 1000. In some implementations, the wearable system 3000 can include the wearable device 3100 and the electronic device 1200. As shown in FIGS. 9A-9B, the wearable device 3100 and the electronic device 1200 can form a unitary structure configured to be secured to the subject 1 (e.g., a wrist of the subject). FIG. 9C illustrates the electronic device 1200 disconnected from the wearable device 3100. Although the figures illustrate implementations of the wearable system 3000 in which the wearable device 3100 and the electronic device 1200 are removably connectable to one another, these components may be integrally formed with one another. For example, in some variants, the wearable device 3100 and electronic device 1200 are integrally formed.

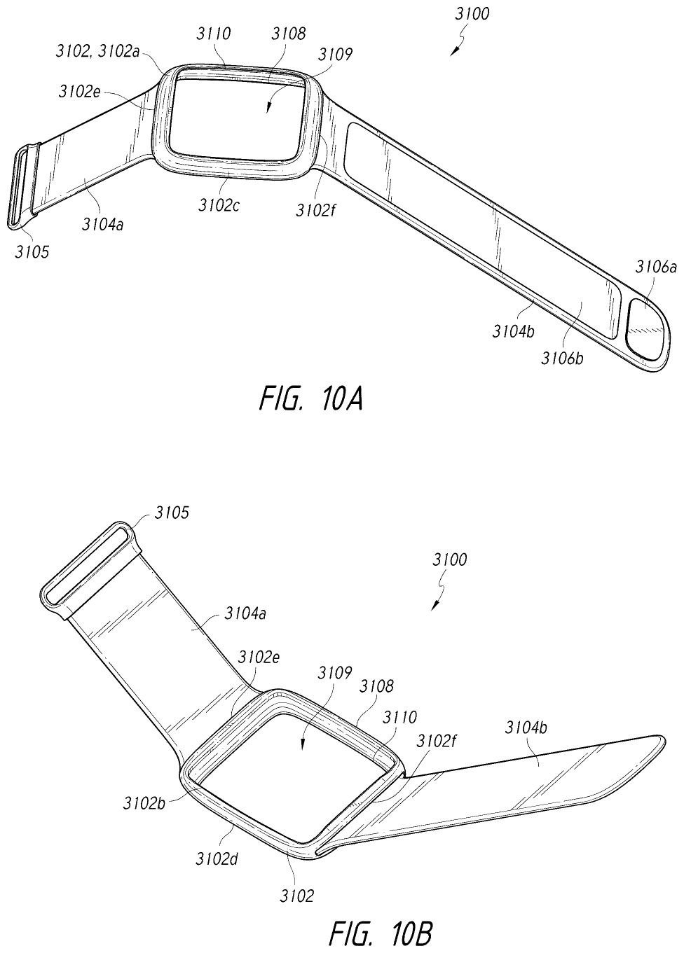

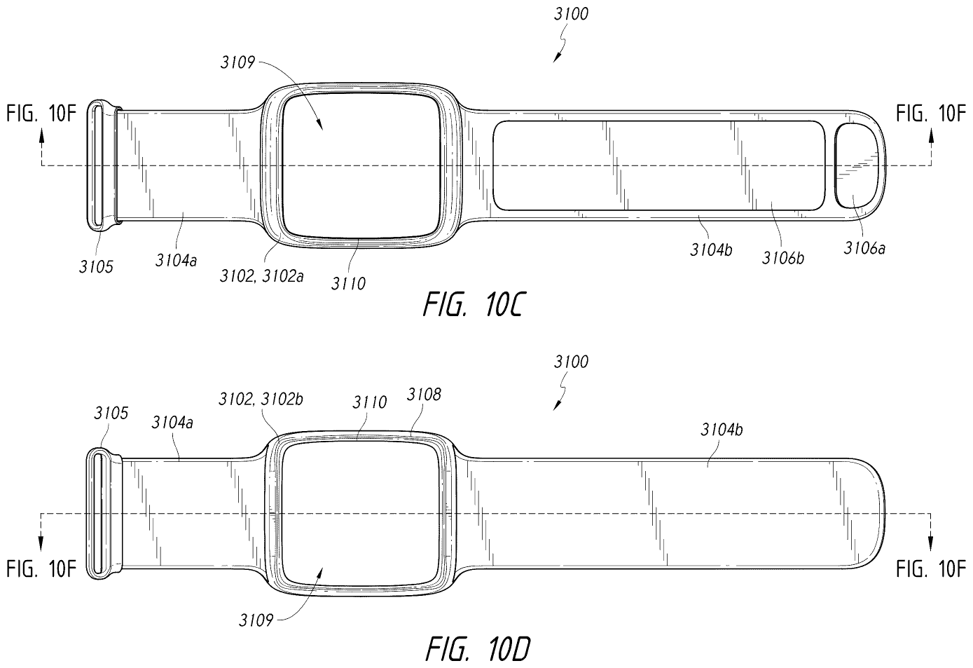

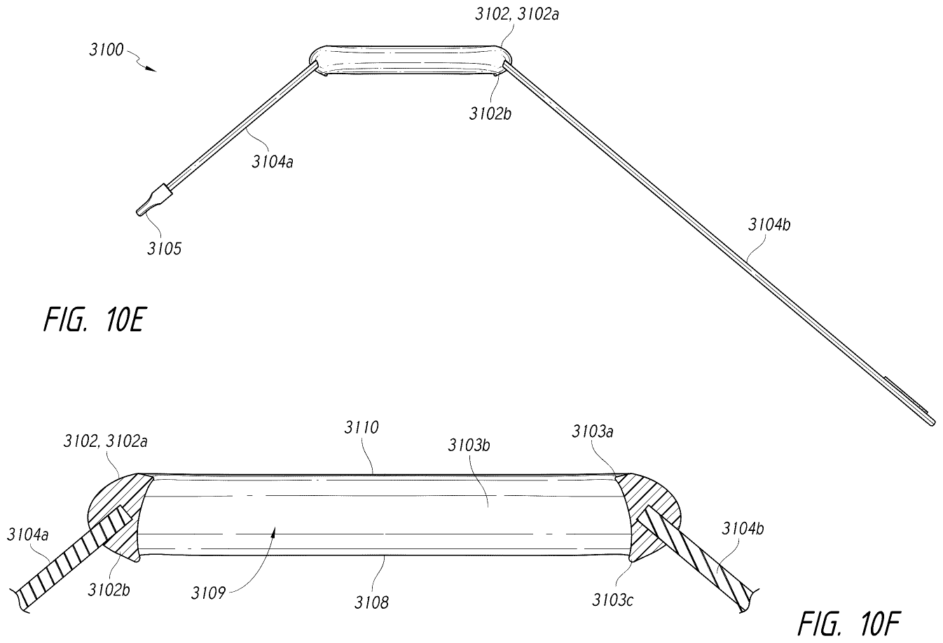

FIGS. 10A-10F illustrate various views of the wearable device 3100 of the wearable system 3000. FIG. 10A illustrates a top perspective view, FIG. 10B illustrates a bottom perspective view, FIG. 10C illustrates a top view, FIG. 10D illustrates a bottom view, FIG. 10E illustrates a side view, and FIG. 10F illustrates a cross-sectional side view of a portion of the wearable device 3100 as indicated in FIGS. 10C-10D. The wearable device 3100 can be similar to the wearable device 1100 and can include any or all features and/or functionality of the wearable device 1100. For example, the wearable device 3100 can include a body portion 3102 and a securement portion connected to the body portion 3102 which can be similar to the body portion 1102 and securement portion connected thereto described with respect to the wearable device 1100. Further to this example, the body portion 3102 can include a first side 3102a, a second side 3102b opposite the first side 3102a, ends 3102c, 3102d, ends 3102e, 3102f, a cavity 3109, and an opening 3108 which can be similar to the body portion 1102, first side 1102a, second side 1102b, ends 1102c, 1102d, ends 1102e, 1102f, cavity 1109, and opening 1108 of the wearable device 1100. Furthermore, the wearable device 3100 can include a securement portion including straps 3104a, 3104b, loop 3105, and attachment portions 3106a, 3106b that are similar or identical to the straps 1104a, 1104b, loop 1105, and attachment portions 1106a, 1106b of wearable device 1100. The wearable device 3100 can differ from the wearable device 1100 in that it can have a different configuration of the body portion 3102 as described further below.

As shown in FIGS. 10A-10F, the body portion 3102 of the wearable device 3100 can include a resilient material configured to removably secure the electronic device 1200. In some implementations, the body portion 3102 can be similar or the same and can include any or all functionality and/or features as the frame 1103 of the wearable device 1100. For example, the body portion 3102 can include engagement features configured to retain and/or position the electronic device 1200 within the wearable device 3100 (e.g., at least partially within cavity 3109). As shown in FIG. 10F, the body portion 3102 can have an upper ridge 3103a, a lower ridge 3103c, and an inner portion 3103b spanning between the upper ridge 3103a and the lower ridge 3103c. The upper ridge 3103a can be a continuous or a discontinuous ridge along a top portion of the body portion 3102 that prevents/limits further upward movement of the electronic device 1200 within the body portion 3102 when the electronic device 1200 and wearable device 3100 are secured to one another. For example, a portion of the top 1292 and/or an upper portion of the side 1296 of the electronic device 1200 can seat against the upper ridge 3103a when the electronic device 1200 and wearable device 3100 are secured to one another. The inner portion 3103b of the body portion 3102 can be configured to receive the side 1296 of the electronic device 1200 when the electronic device 1200 and wearable device 3100 are secured to one another. The lower ridge 3103c of the body portion 3102 can be a continuous or discontinuous ridge along a bottom portion of the body portion 3102 that allows the electronic device 1200 to be removably received by the body portion 3102 and secures the electronic device 1200 within the body portion 3102 when the electronic device 1200 and wearable device 3100 are secured to one another. For example, a portion of the bottom 1294 and/or a lower portion of the side 1296 of the electronic device 1200 can seat against the lower ridge 3103c of the body portion 3102 when the electronic device 1200 and wearable device 3100 are secured to one another. In some implementations, the releasable connection between the body portion 3102 and the electronic device 1200 is a snap fit.

Additionally, and as shown in FIGS. 10A-10F, in some implementations the wearable device 3100 can include an opening 3110. The opening 3110 can be in the first side 3102a. Such an opening can allow a subject access a portion of the electronic device 1200 when the electronic device 1200 is secured by the wearable device 3100 (e.g., at least a portion of the top 1292). Furthermore, such an opening can allow a subject to visibly see the electronic device 1200 when the electronic device 1200 is secured by the wearable device 3100 (e.g., at least a portion of the top 1292). The opening 3110 can facilitate removal of the electronic device 1200 from the wearable device 3100.

The securement portion of the wearable device 3100 including straps 3104a, 3104b can connect to the ends 3102e, 3102f, respectively. In some implementations, the straps 3104a, 3104b can comprise multiple layers similar to or the same as the straps 1104a, 1104b in some implementations of the wearable device 1100. In some implementations, straps 3204a, 3104b comprise silicone and/or silicone rubber. In some implementations, straps 3104a, 3104b comprise fabric.

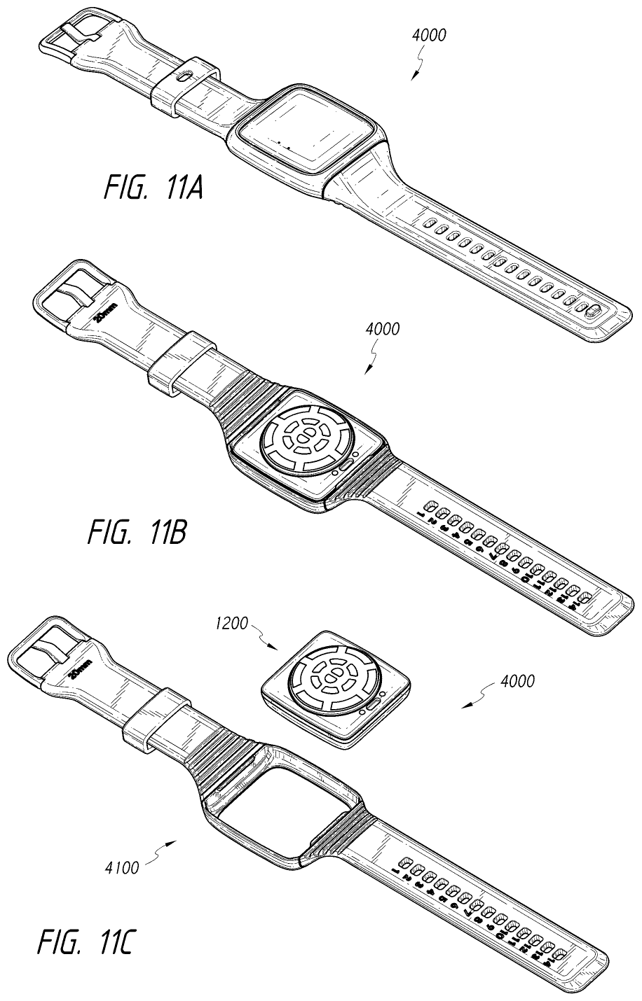

FIGS. 11A-11C illustrate various views of another implementation of a wearable system 4000. The wearable system 4000 can be similar to the wearable systems 1000, 2000, and/or 3000 and can include any or all features and/or functionality of the wearable systems 1000, 2000, and/or 3000. The wearable system 4000 can include a wearable device 4100. The wearable device 4100 can be configured to receive, position, and/or at least partially cover the electronic device 1200 including one or more sensors for measuring one or more physiological parameters of the subject 1 similar or the same as the wearable devices 1100, 2100, and/or 3100 of wearable systems 1000, 2000, and/or 3000. In some implementations, the wearable system 4000 can include the wearable device 4100 and the electronic device 1200. As shown in FIGS. 11A-11B, the wearable device 4100 and the electronic device 1200 can form a unitary structure configured to be secured to the subject 1 (e.g., a wrist of the subject). FIG. 11C illustrates the electronic device 1200 disconnected from the wearable device 4100. Although the figures illustrate implementations of the wearable system 4000 in which the wearable device 4100 and the electronic device 1200 are removably connectable to one another, these components may be integrally formed with one another. For example, in some variants, the wearable device 4100 and electronic device 1200 are integrally formed.

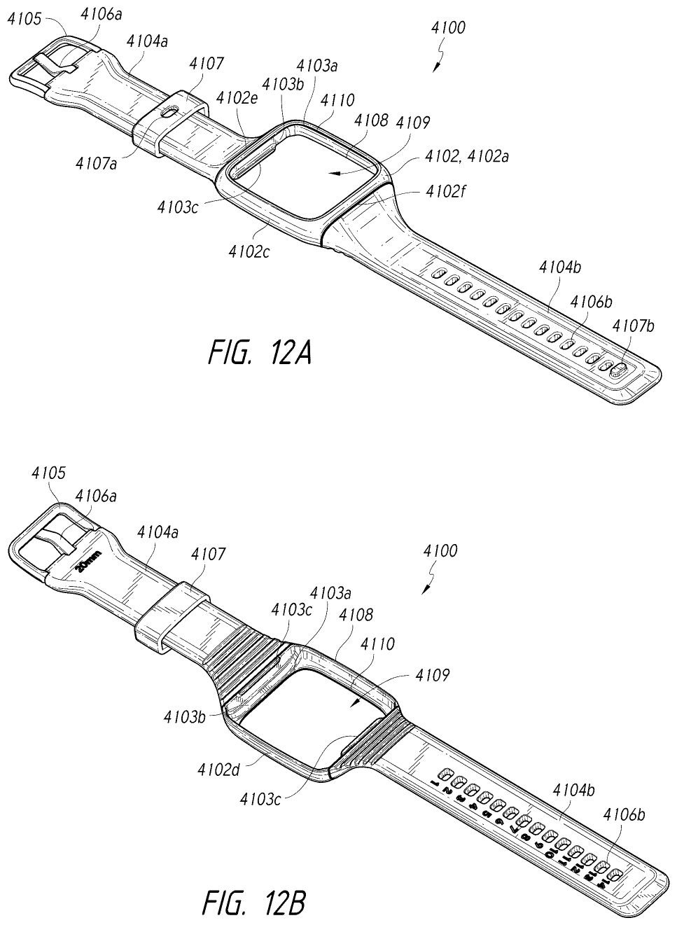

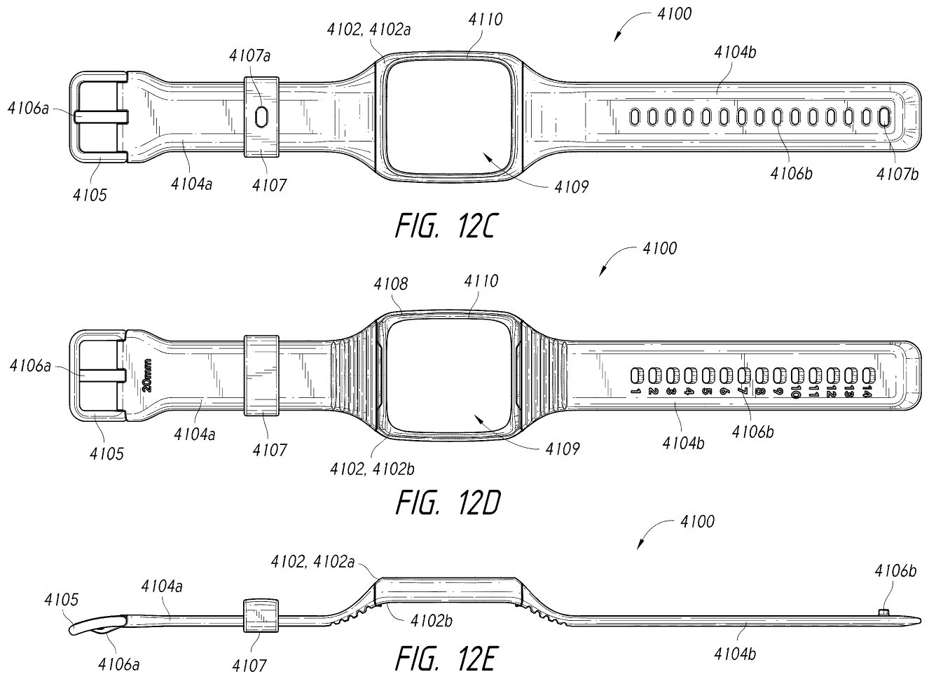

FIGS. 12A-12E illustrate various views of the wearable device 4100 of the wearable system 4000. FIG. 12A illustrates a top perspective view, FIG. 12B illustrates a bottom perspective view, FIG. 12C illustrates a top view, FIG. 12D illustrates a bottom view, and FIG. 12E illustrates a side view of the wearable device 4100. The wearable device 4100 can be similar to the wearable devices 1100, 2100, and/or 3100 and can include any or all features and/or functionality of the wearable devices 1100, 2100, and/or 3100. For example, the wearable device 4100 can include a body portion 4102 and a securement portion connected to the body portion 4102 which can be similar to the body portions 1102, 2102, and/or 3102 and the respective securement portions connected thereto described with respect to the wearable devices 1100, 2100, and/or 3100. Further to this example, the body portion 4102 can include a first side 4102a, a second side 4102b opposite the first side 4102a, ends 4102c, 4102d, ends 4102e, 4102f, a cavity 4109, and an opening 4108 which can be similar to corresponding features of the wearable devices 1100, 2100, and/or 3100. Furthermore, the wearable device 4100 can have an opening 4110 which can be similar to the opening 3110 of the wearable device 3100.

The wearable device 4100 can be similar to the wearable device 2100 in that it can be a formed (e.g., molded) structure. Such structure can comprise a silicone and/or a silicone rubber. Furthermore, such structure can be without multiple layers. The wearable device 4100 can also be similar to the wearable device 2100 in that it can be without a frame (e.g., the body portion 4102 may not include a frame within cavity 4109). In such implementations, the body portion 4102 can be configured to position and secure the electronic device 1200 without such a frame. For example, the body portion 4102 can include engagement features configured to retain and/or position the electronic device 1200 within the wearable device 4100 (e.g., at least partially within cavity 4109). As shown in FIGS. 12A-12B, such engagement features can include an upper ridge 4103a, a lower ridge 4103c, and an inner portion 4103b spanning between the upper ridge 4103a and the lower ridge 4103c. Such engagement features can be similar or the same and/or include any or all functionality as the engagement features of wearable device 3100 such as upper ridge 3103a, lower ridge 3103a, and inner portion 3103b. In some variants, the wearable device 4100 can include a frame similar or identical to the frame 1103 of wearable device 1100.

The wearable device 4100 can differ from the wearable devices 1100, 2100, and/or 3100 in that it can have a different securement portion configuration. As shown in FIGS. 12A-12E, the securement portion including straps 4104a, 4104b can have similar or the same features and/or functionality as the securement portion including straps 2104a, 2104b. The securement portion of wearable device 4100 can include a loop 4105, for example, disposed at an end of strap 4104a and configured to receive an end of strap 4104b for securing the straps 4104a, 4104b to one another. In addition, the securement portion of wearable device 4100 can include attachment portions configured to allow the straps 4104a, 4104b to secure to one another. Such attachment portions can include a tongue 4106a, which can combine with loop 4105 to form a buckle and can be disposed at an end of strap 4104a, and openings 4106b, which can be disposed along at least a portion of a length of strap 4104b. The openings 4106b can be configured to receive the tongue 4106a therethrough. Furthermore, in some implementations the securement portion can include additional attachment portions including a loop 4107, which can be disposed about strap 4104a and configured to receive an end of the strap 4104b therethrough. The loop 4107 can include an opening 4107a configured to receive a corresponding protrusion 4107b disposed on strap 4104b for further securement of the straps 4104a, 4104b to one another.

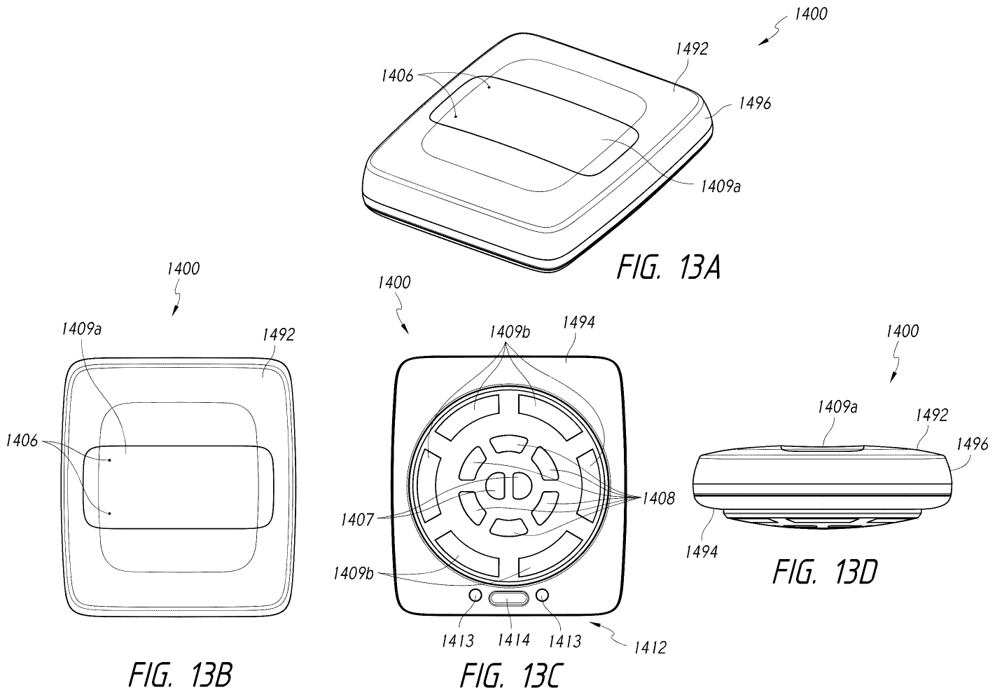

FIG. 13A-13D illustrate various views of an electronic device 1400 that can be incorporated into any of the wearable devices described herein. FIG. 13A shows a top perspective view, FIG. 13B shows a top view, FIG. 13C shows a bottom view, and FIG. 13D shows a side view of the electronic device 1400. The electronic device 1400 is identical to the electronic device 1200 and includes all features and/or functionality of the electronic device 1200 except that its one or more ECG electrodes 1409a, 1409b, when included, can have a different configuration than the one or more ECG electrodes 1209 of the electronic device 1200. For example, the electronic device 1400 can have a top 1492, a bottom 1494, a side 1496, and an overall shape that is identical to the top 1292, the bottom 1294, the side 1296, and the overall shape of the electronic device 1200. Furthermore, the electronic device 1400 can have one or more status indicators 1406, one or more emitters 1407, and one or more detectors 1408 that are identical to the one or more status indicators 1206, the one or more emitters 1207, and the one or more detectors 1208 of the electronic device 1200. The electronic device 1400 can also have a charge port 1412 including electrical contacts 1413 and a feature 1414 that are identical to the charge port 1212 including electrical contacts 1213 and the feature 1214 of the electronic device 1200. Additionally, any of the features of the electronic device 1200 that are described with respect to FIG. 2F can be included in the electronic device 1400. In an implementation of the different ECG electrode configuration and as shown in FIGS. 13A-13C, the one or more ECG electrodes of electronic device 1400 can include one or more ECG electrodes 1409b that can be similar or identical to the one or more ECG electrodes 1209 of electronic device 1200, as well as an ECG electrode 1409a positioned along a portion of the top 1492. In such an implementation, when the subject wants to make a measurement using the ECG sensor that includes the ECG electrodes 1409a, 1409b, the subject can press on or touch the ECG electrode 1409a using a finger or another part of their body such that the subject's skin makes contact with the ECG electrode 1409a. A processor of the electronic device 1400 can determine when the subject touches the ECG electrode 1409a and utilize the ECG electrodes 1409a, 1409b to measure (for example, automatically measure) an ECG of the subject.

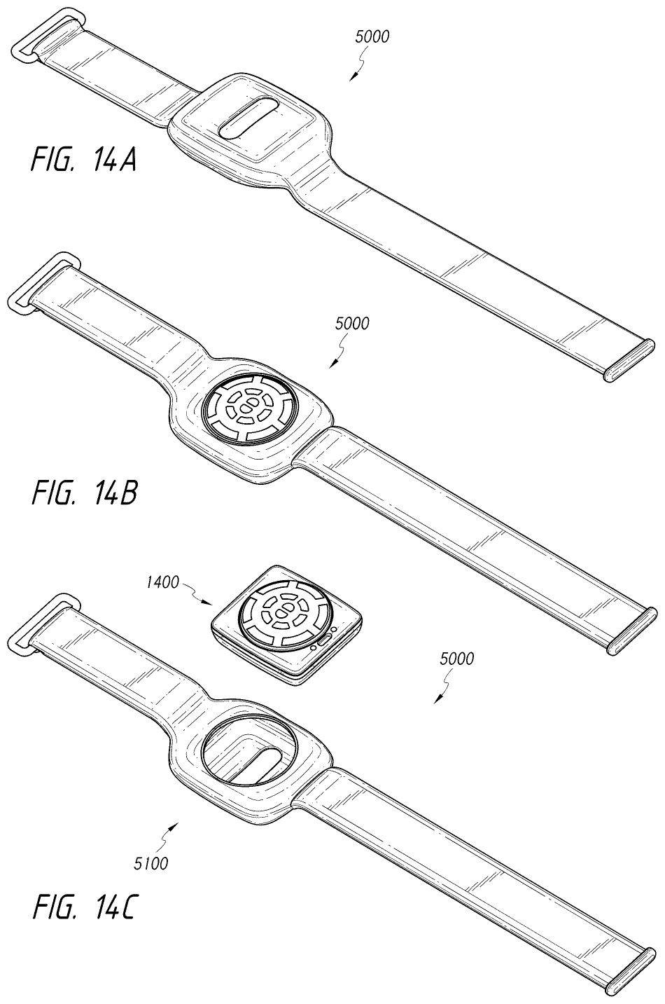

FIGS. 14A-14C illustrate various views of another implementation of a wearable system 5000. The wearable system 5000 can be similar to the wearable systems 1000, 2000, 3000 and/or 4000 and can include any or all features and/or functionality of the wearable systems 1000, 2000, 3000 and/or 4000. The wearable system 5000 can include a wearable device 5100. The wearable device 5100 can be configured to receive, position, and/or at least partially cover the electronic device 1400 including one or more sensors for measuring one or more physiological parameters of the subject 1 similar or the same as the wearable devices 1100, 2100, 3100 and/or 4100 of wearable systems 1000, 2000, 3000 and/or 4000. In some implementations, the wearable system 5000 can include the wearable device 5100 and the electronic device 1400. As shown in FIGS. 14A-14B, the wearable device 5100 and the electronic device 1400 can form a unitary structure configured to be secured to the subject 1 (e.g., a wrist of the subject). FIG. 14C illustrates the electronic device 1400 disconnected from the wearable device 5100. Although the figures illustrate implementations of the wearable system 5000 in which the wearable device 5100 and the electronic device 1400 are removably connectable to one another, these components may be integrally formed with one another. For example, in some variants, the wearable device 5100 and electronic device 1400 are integrally formed.

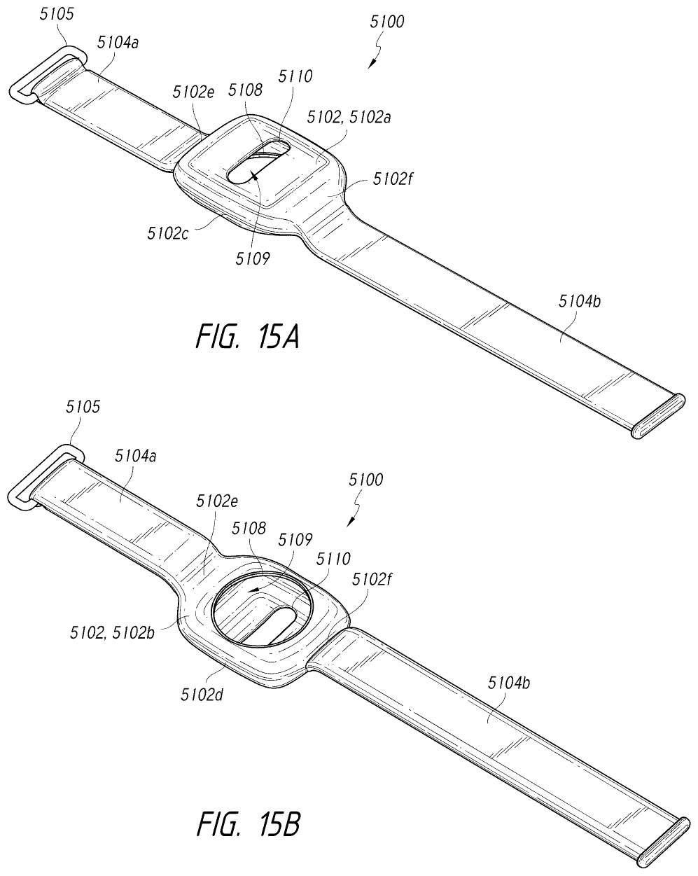

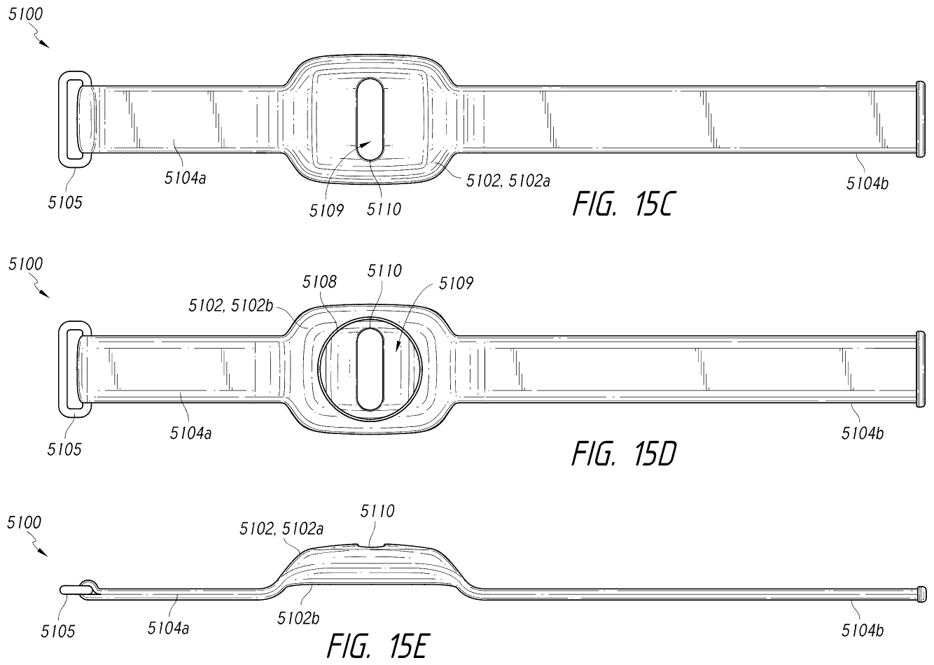

FIGS. 15A-15E illustrate various views of the wearable device 5100 of the wearable system 5000. FIG. 15A illustrates a top perspective view, FIG. 15B illustrates a bottom perspective view, FIG. 15C illustrates a top view, FIG. 15D illustrates a bottom view, and FIG. 15E illustrates a side view of the wearable device 5100. The wearable device 5100 can be similar to the wearable devices 1100, 2100, 3100 and/or 4100 and can include any or all features and/or functionality of the wearable devices 1100, 2100, 3100 and/or 4100. For example, the wearable device 5100 can include a body portion 5102 and a securement portion connected to the body portion 5102 which can be similar to the body portions 1102, 2102, 3102 and/or 4102 and the respective securement portions connected thereto described with respect to the wearable devices 1100, 2100, 3100 and/or 4100. Further to this example, the body portion 5102 can include a first side 5102a, a second side 5102b opposite the first side 5102a, ends 5102c, 5102d, ends 5102e, 5102f, a cavity 5109, and an opening 5108 which can be similar to corresponding features of the wearable devices 1100, 2100, 3100 and/or 4100. Furthermore, the wearable device 5100 can have an opening 5110 which can be similar to the openings 3110, 4110 of the wearable devices 3100, 4100. The opening 5110 can be positioned in the first side 5102a and configured such that it allows a subject to utilize (e.g., touch) ECG electrode 1409a of electronic device 1400 when the electronic device 1400 is secured by the wearable device 5100. In some implementations and as shown, the opening 5110 can be smaller than the opening 5108. In some implementations (not shown), the opening 5110 can be larger than the opening 5108.

The wearable device 5100 can be similar to the wearable device 1100 in that it can comprise a fabric material. In some implementations, the wearable device 5100 can include more than one layer similar or the same as in some implementations of wearable device 1100. In some variants, the wearable device 5100 can be a formed (e.g., molded) structure. Such structure can comprise a silicone and/or a silicone rubber. Furthermore, such structure can be without multiple layers. The wearable device 5100 can be similar to the wearable device 2100 in that it can be without a frame (e.g., the body portion 5102 may not include a frame within cavity 5109). In such implementations, the body portion 5102 can be configured to position and secure the electronic device 1400 without such a frame.

The wearable device 5100 can differ from the wearable devices 1100, 2100, 3100 and/or 4100 in that it can have a different securement portion configuration. As shown in FIGS. 15A-15E, the securement portion including straps 5104a, 5104b can have similar or the same features and/or functionality as any of the straps described herein. The securement portion of wearable device 5100 can include a loop 5105, for example, disposed at an end of strap 5104a and configured to receive an end of strap 5104b for securing the straps 5104a, 5104b to one another. In addition, the securement portion of wearable device 5100 can include attachment portions configured to allow the straps 5104a, 5104b to secure to one another. Such attachment portions can be integrated within straps 5104a, 5104b and can include a magnetic attachment and/or a hook-and-loop type attachment.

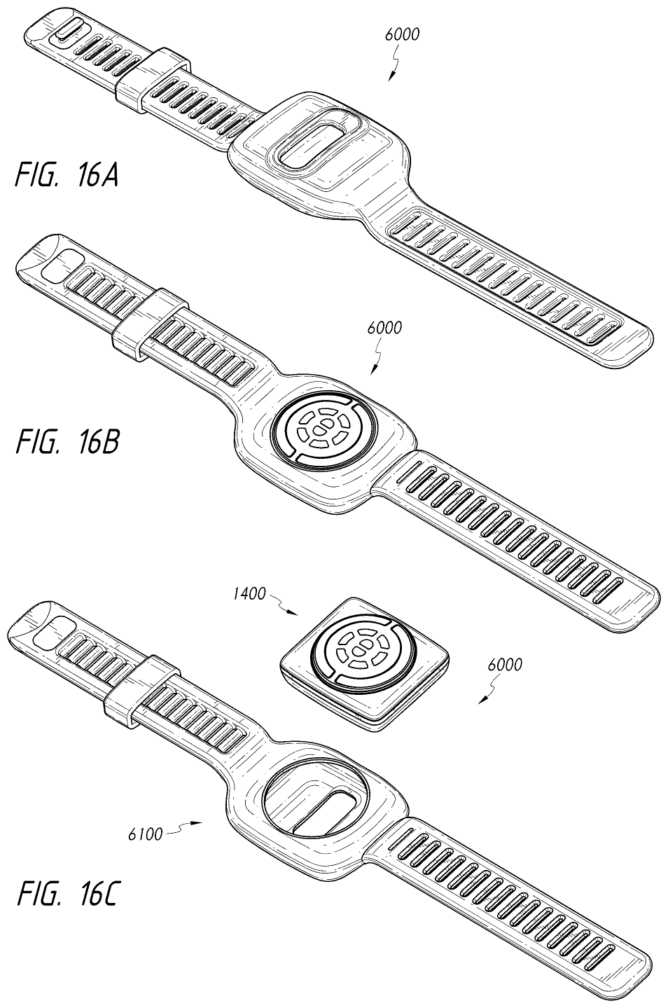

FIGS. 16A-16C illustrate various views of another implementation of a wearable system 6000. The wearable system 6000 can be similar to the wearable system 2000 and can include any or all features and/or functionality of the wearable system 2000. The wearable system 6000 can include a wearable device 6100. The wearable device 6100 can be configured to receive, position, and/or at least partially cover the electronic device 1400 including one or more sensors for measuring one or more physiological parameters of the subject 1 similar or the same as any of the wearable devices described herein. In some implementations, the wearable system 6000 can include the wearable device 6100 and the electronic device 1400. As shown in FIGS. 16A-16B, the wearable device 6100 and the electronic device 1400 can form a unitary structure configured to be secured to the subject 1 (e.g., a wrist of the subject). FIG. 16C illustrates the electronic device 1400 disconnected from the wearable device 6100. Although the figures illustrate implementations of the wearable system 6000 in which the wearable device 6100 and the electronic device 1400 are removably connectable to one another, these components may be integrally formed with one another. For example, in some variants, the wearable device 6100 and electronic device 1400 are integrally formed.

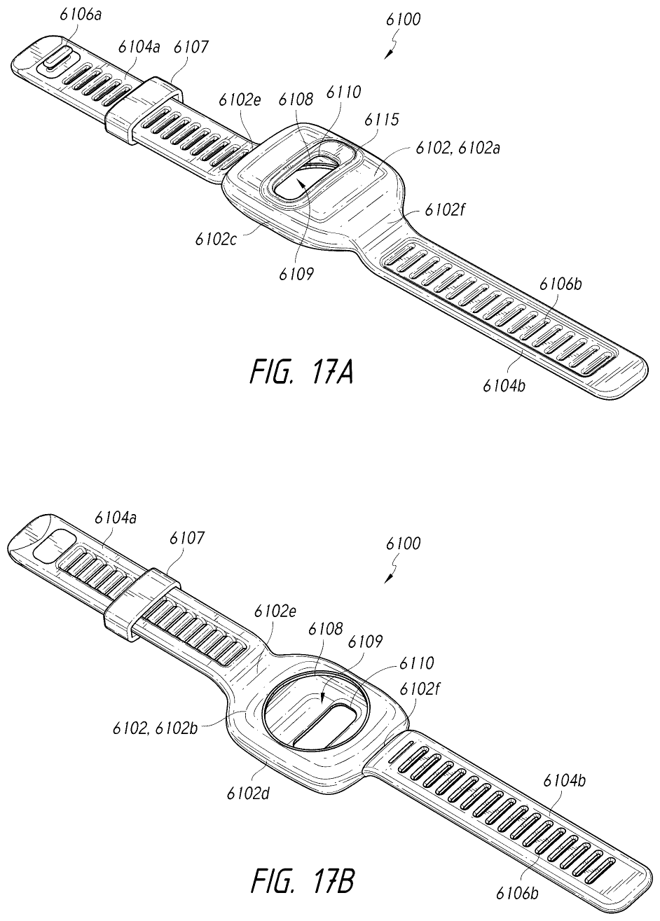

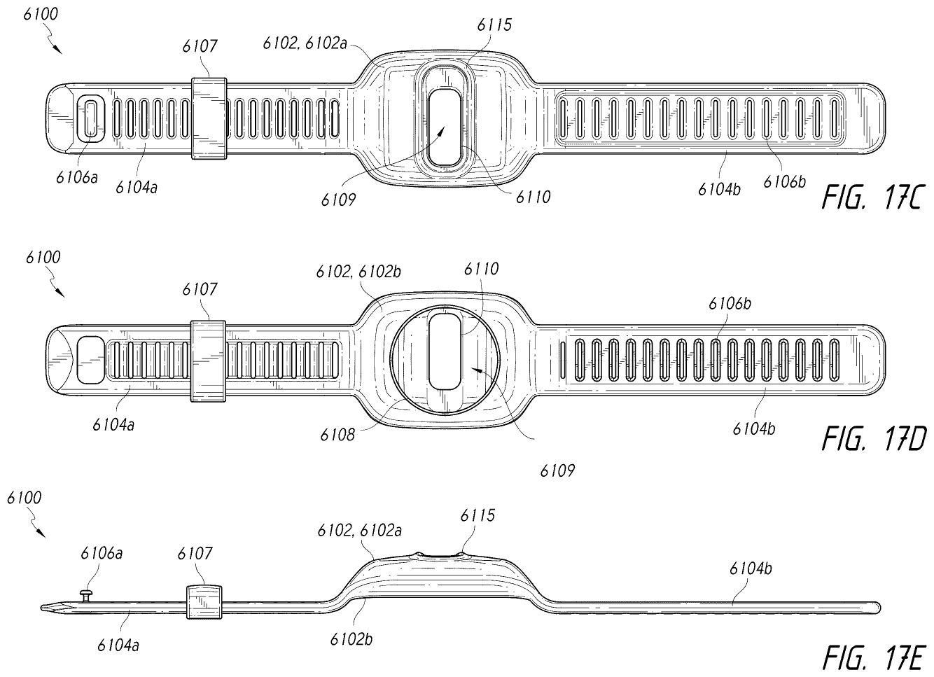

FIGS. 17A-17E illustrate various views of the wearable device 6100 of the wearable system 6000. FIG. 17A illustrates a top perspective view, FIG. 17B illustrates a bottom perspective view, FIG. 17C illustrates a top view, FIG. 17D illustrates a bottom view, and FIG. 17E illustrates a side view of the wearable device 6100. The wearable device 6100 can be identical to the wearable device 2100 and can include any or all features and/or functionality of the wearable device 2100 except it can have a different configuration of its body portion 6102 described further below. The wearable device 6100 can include a body portion 6102 and a securement portion connected to the body portion 6102 which can be similar or identical to the body portion 2102 and securement portion connected thereto described with respect to the wearable device 2100. The body portion 6102 can include a first side 6102a, a second side 6102b opposite the first side 6102a, ends 6102c, 6102d, ends 6102e, 6102f, a cavity 6109, and an opening 6108 which can be similar or identical to the body portion 2102, first side 2102a, second side 2102b, ends 2102c, 2102d, ends 2102e, 2102f, cavity 2109, and opening 2108 of the wearable device 2100. The wearable device 6100 can differ from the wearable device 2100 in that it can have an opening 6110. Such opening 6110 can be similar to the openings 3110, 4110 of the wearable devices 3100, 4100. The opening 6110 can be positioned in the first side 6102a and configured such that it allows a subject to utilize (e.g., touch) ECG electrode 1409a of electronic device 1400 when the electronic device 1400 is secured by the wearable device 6100. In some implementations and as shown, the opening 6110 can be smaller than the opening 6108. In some implementations (not shown), the opening 6110 can be larger than the opening 6108. Furthermore, and as shown, in some implementations the wearable device 6100 can differ from the wearable device 2100 in that it can have a ridge 6115. Such ridge 6115 can extend upwards from the first side 6102a and at least partially surround the opening 6110. Such ridge 6115 can advantageously aid in a subject locating the opening 6110 for obtaining an ECG measurement via electronic device 1400.

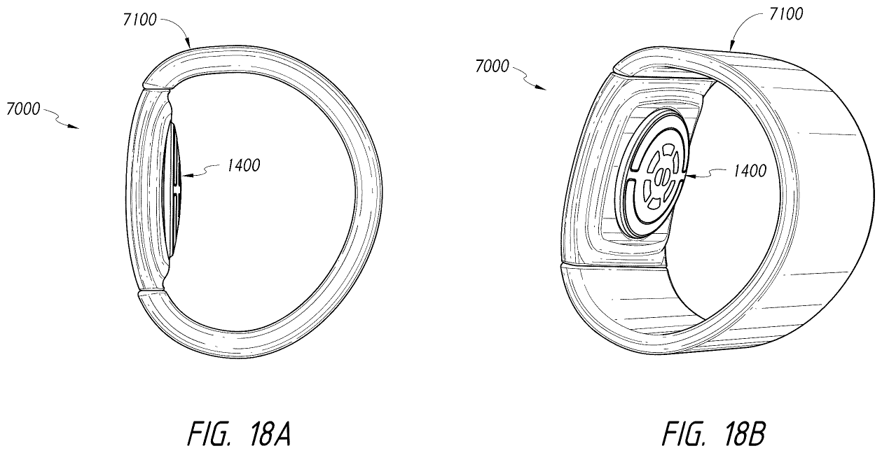

FIGS. 18A-18B illustrate various views of another implementation of a wearable system 7000. The wearable system 7000 can be similar to any of the wearable systems described herein and can include any or all features and/or functionality of the wearable systems described herein. The wearable system 7000 can include a wearable device 7100. The wearable device 7100 can be configured to receive, position, and/or at least partially cover the electronic device 1400 including one or more sensors for measuring one or more physiological parameters of the subject 1 similar or the same as any of the wearable devices described herein. In some implementations, the wearable system 7000 can include the wearable device 7100 and the electronic device 1400. As shown in FIGS. 18A-18B, the wearable device 7100 and the electronic device 1400 can form a unitary structure configured to be secured to the subject 1 (e.g., a wrist of the subject). In some implementations, the electronic device 1400 and the wearable device 7100 are configured to be removably securable to one another. In some implementations, the electronic device 1400 and the wearable device 7100 may be integrally formed with one another.

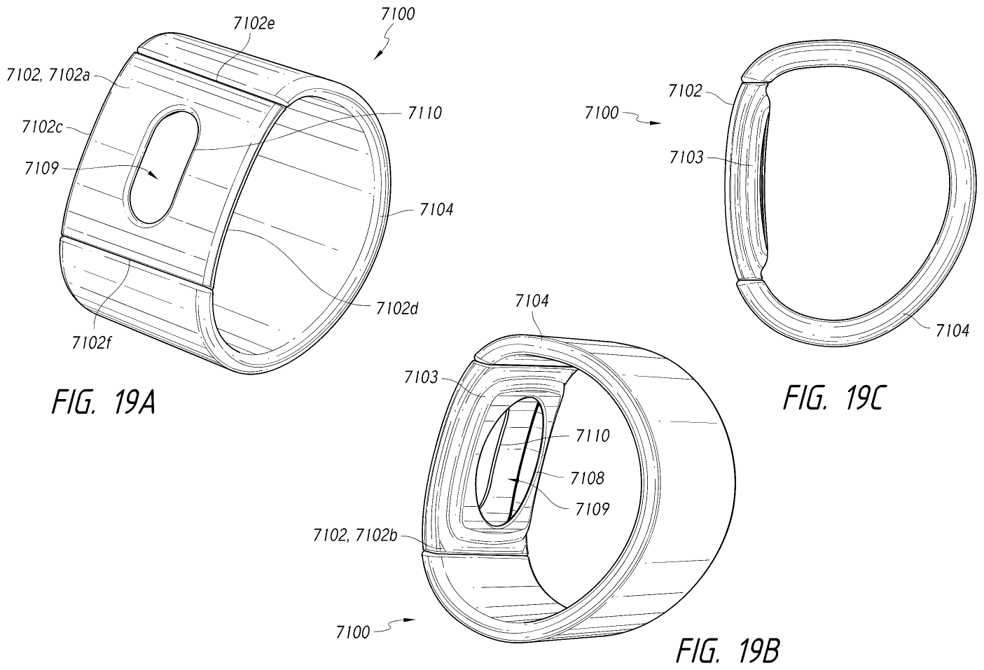

FIGS. 19A-19C illustrate various views of the wearable device 7100 (which can also be referred to herein as a “band”) of the wearable system 7000. FIGS. 19A-19B illustrate perspective views and FIG. 19C illustrates a side view of the wearable device 7100. The wearable device 7100 can be similar to any of the wearable devices described herein and can include any or all features and/or functionality of the wearable devices described herein. For example, the wearable device 7100 can include a body portion 7102 and a securement portion connected to the body portion 7102 which can be similar to any of the body portions and/or securement portions described herein. Further to this example, the body portion 7102 can include a first side 7102a, a second side 7102b opposite the first side 7102a, ends 7102c, 7102d, ends 7102e, 7102f, a cavity 7109, an opening 7108, and an opening 7710 which can be similar to any of such corresponding features (which can begin with a different number) described herein. The wearable device 7100 can differ from other wearable devices described herein in that the securement portion can include a strap 7104 that is connected at each of its ends to the body portion 7102 at its ends 7102e, 7102f as shown. In some implementations, the connection between strap 7104 and body portion 7102 is seamless. In some implementations, the strap 7104 can be integrally formed with at least a portion of the body portion 7102 (e.g., from the same material). In some implementations, at least a portion of the body portion 7102 and/or the strap 7104 can comprise a flexible, stretchy, and/or resilient material. In some implementations, wearable device 7100 or portions thereof can comprise a material with sufficient elasticity to allow the wearable device 7100 to extend such that it can pass over a hand and contract to a size to be snug around a wrist of the subject.

In some implementations, the body portion 7102 can include a frame 7103 which can function similarly or the same as the frames described herein. For example, the frame 7103 can comprise a material that is more rigid than a material from which the body portion 7102 and/or the strap 7104 is made. In some implementations, the frame 7103 can comprise a flexible and/or resilient material. Furthermore, in some implementations, the frame 7103 can include the opening 7108 for receiving therethrough and positioning the electronic device 1400 within the cavity 7109. In some variants of the wearable device 7100, the body portion 7102 can include a slit in addition to or in lieu of the opening 7108 for receiving the electronic device 1400 for positioning within the cavity 7109.

Similar to the openings 5110, 6110 of the wearable devices 5100, 6100, the opening 7110 in the first side 7102a of the body portion 7102 can at least partially align with the ECG electrode 1490a of electronic device 1400 when secured by the wearable device 7100 and allow a subject to access the ECG electrode 1490a for taking at least an ECG measurement. In some implementations, opening 7110 of the first side 7102a is smaller than the opening 7108 of the second side 7102b. In some implementations, opening 7110 of the first side 7102a is larger than the opening 7108 of the second side 7102b.

In some implementations and as shown, the body portion 7102 of the wearable device 7100 can have a width (e.g., extending between ends 7102c, 7102d) that is substantially similar or the same as a width of the securement portion (e.g., a width of the strap 7104). In variants of the wearable device 7100, the body portion 7102 can have a width (e.g., extending between ends 7102c, 7102d) that is greater than a width of the securement portion (e.g., a width of the strap 7104).

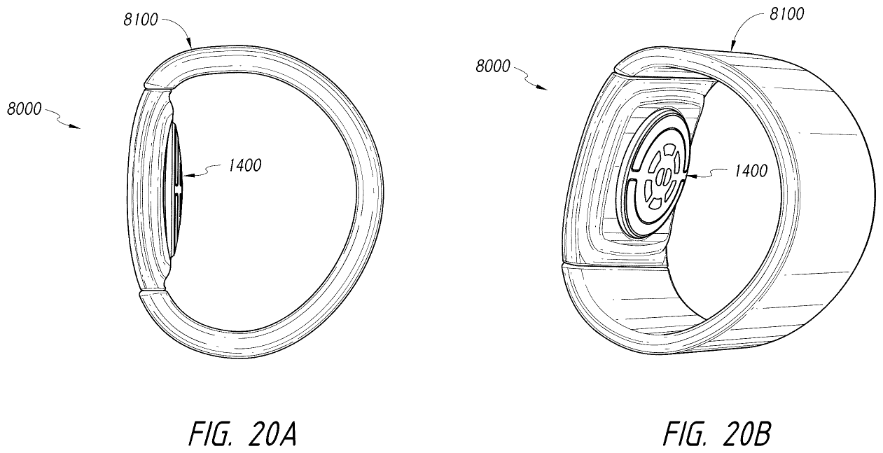

FIGS. 20A-20B illustrate various views of another implementation of a wearable system 8000. The wearable system 7000 can be similar to the wearable system 8000 and/or any of the wearable systems described herein and can include any or all features and/or functionality of the wearable system 8000 and/or any of the wearable devices described herein. The wearable system 8000 can include a wearable device 8100. The wearable device 8100 can be configured to receive, position, and/or at least partially cover the electronic device 1400 including one or more sensors for measuring one or more physiological parameters of the subject 1 similar or the same as any of the wearable devices described herein. In some implementations, the wearable system 8000 can include the wearable device 8100 and the electronic device 1400. As shown in FIGS. 20A-20B, the wearable device 8100 and the electronic device 1400 can form a unitary structure configured to be secured to the subject 1 (e.g., a wrist of the subject). In some implementations, the electronic device 1400 and the wearable device 8100 are configured to be removably securable to one another. In some implementations, the electronic device 1400 and the wearable device 8100 may be integrally formed with one another.

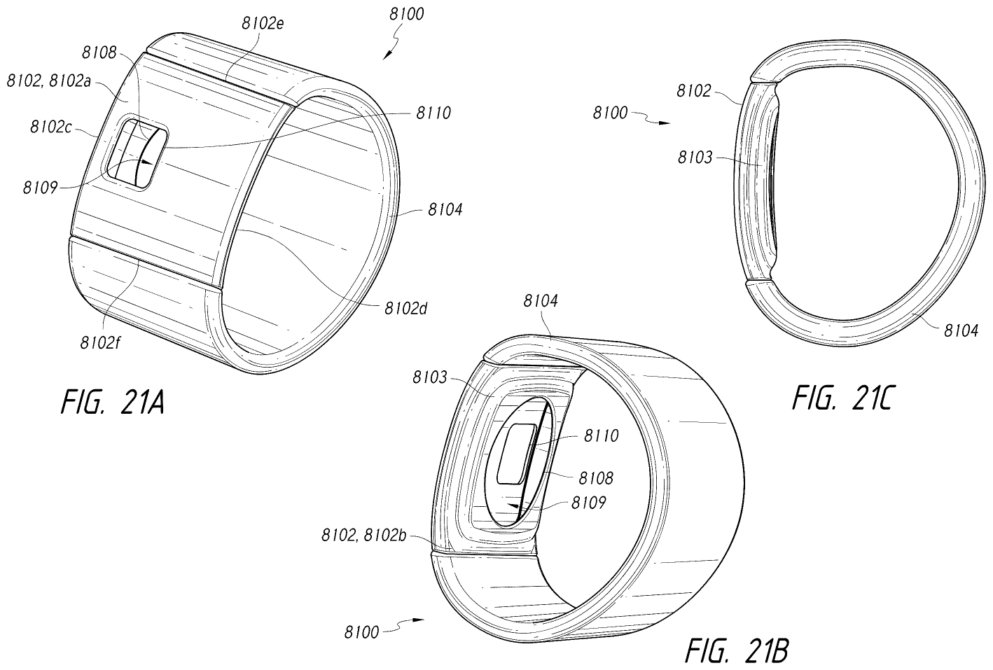

FIGS. 21A-21C illustrate various views of the wearable device 8100 (which can also be referred to herein as a “band”) of the wearable system 8000. FIGS. 21A-21B illustrate perspective views and FIG. 21C illustrates a side view of the wearable device 8100. The wearable device 8100 can be identical to the wearable device 7100 and can include any or all features and/or functionality of the wearable device 7100 except that it can have a different configuration of its corresponding opening 8110. For example, the wearable device 8100 can include a body portion 8102 including a first side 8102a, a second side 8102b opposite the first side 8102a, ends 8102c, 8102d, ends 8102e, 8102f, a cavity 8109, an opening 8108, a securement portion including a strap 8104, and a frame 8103 that can be identical to corresponding features (which can begin with a different number) of the wearable device 7100 except for the opening 8110.

Similar to the openings 7110 of the wearable device 7100, the opening 8110 in the first side 8102a of the body portion 8102 can at least partially align with the ECG electrode 1490a of electronic device 1400 when secured by the wearable device 8100 and allow a subject to access the ECG electrode 1490a for taking at least an ECG measurement. The opening 8110 can, in some implementations, additionally allow light from status indicators 1406 of the electronic device 1400 to shine therethrough. In some implementations, the wearable device 8100 can receive and position the electronic devices 1200 and/or 1300 described herein. In such implementations, light from corresponding status indicators 1206, 1306 can similarly shine through the opening 8110.

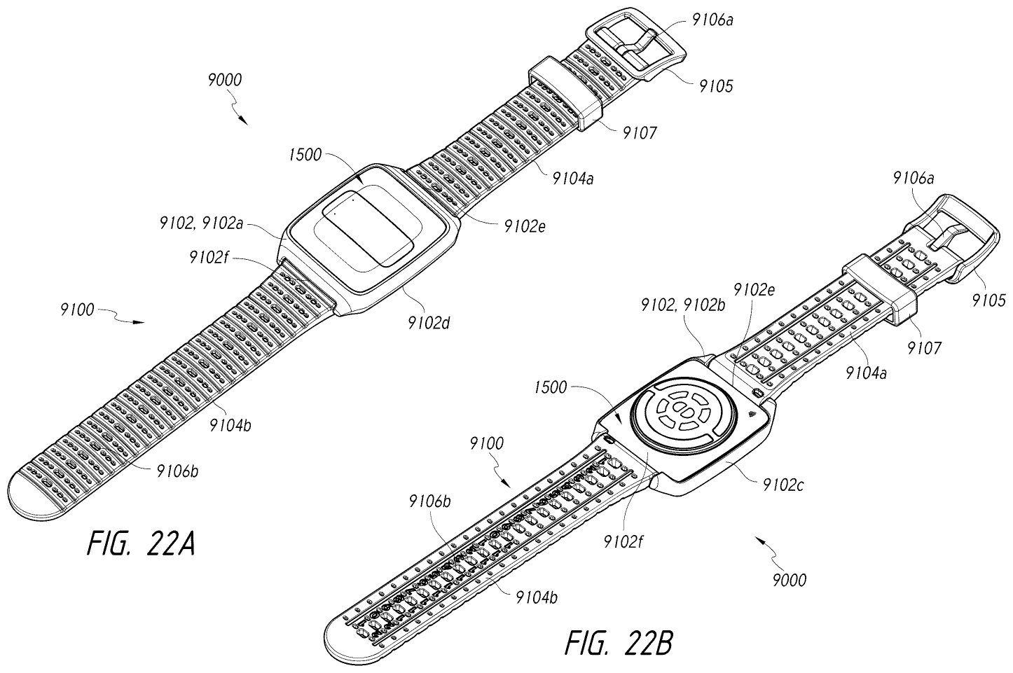

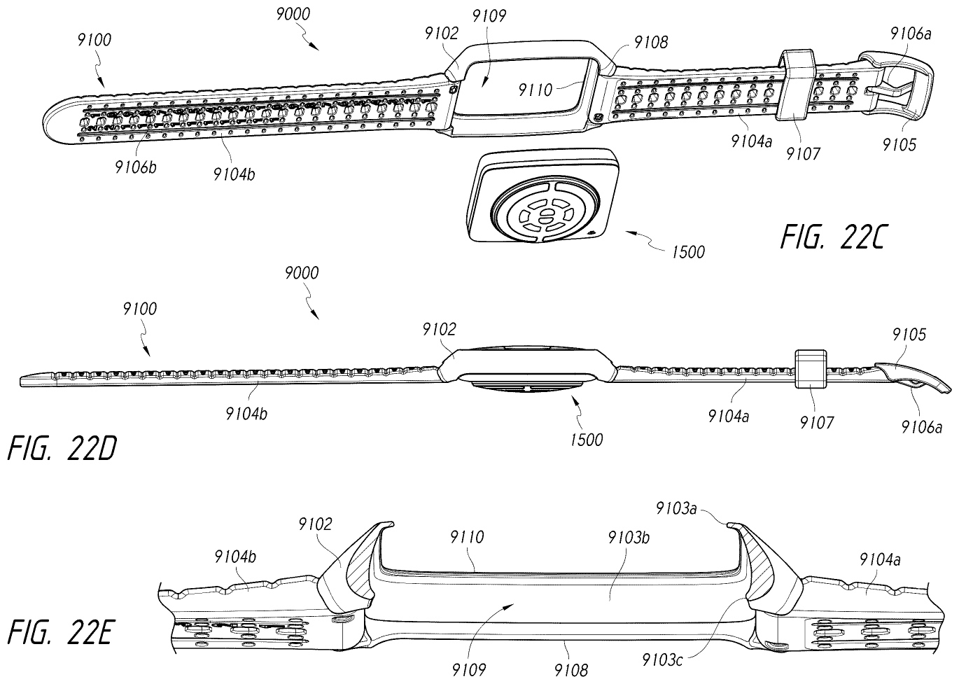

FIGS. 22A-22E illustrate various views of another implementation of a wearable system 9000. The wearable system 9000 can be similar to the wearable system 4000 and/or any of the wearable systems described herein and can include any or all features and/or functionality of the wearable system 4000 and/or any of the wearable devices described herein. The wearable system 9000 can include a wearable device 9100. The wearable device 9100 can be configured to receive, position, and/or at least partially cover the electronic device 1400 including one or more sensors for measuring one or more physiological parameters of the subject 1 similar or the same as any of the wearable devices described herein. In some implementations, the wearable system 9000 can include the wearable device 9100 and the electronic device 1400. As shown in FIGS. 22A-22B & 22D, the wearable device 9100 and the electronic device 1400 can form a unitary structure configured to be secured to the subject 1 (e.g., a wrist of the subject). In some implementations, the electronic device 1400 and the wearable device 9100 are configured to be removably securable to one another such as shown in FIG. 22C. In some implementations, the electronic device 1400 and the wearable device 9100 may be integrally formed with one another.

The wearable device 9100 can be similar to the wearable device 4100 and/or any of the wearable devices described herein and can include any or all features and/or functionality of the wearable device 4100 and/or of any of the wearable devices described herein. For example, the wearable device 9100 can include a body portion 9102 and a securement portion connected to the body portion 9102 which can be similar to the body portion 4102 and/or any of the body portions described herein (which can begin with a different number) and the respective securement portions connected thereto described with respect to the wearable device 4100 and/or any of the wearable devices described herein. Further to this example, the body portion 9102 can include a first side 9102a, a second side 9102b opposite the first side 9102a, ends 9102c, 9102d, ends 9102e, 9102f, a cavity 9109, an opening 9108, which can be similar to corresponding features of the wearable device 4100 or any of the wearable devices described herein (which can begin with a different number). Furthermore, the wearable device 9100 can have an opening 9110 which can be similar to the opening 4110 of the wearable device 4100.

The wearable device 9100 can further be similar to the wearable device 4100 in that it can be a formed (e.g., molded) structure. Such structure can comprise a silicone and/or a silicone rubber. Furthermore, such structure can be without multiple layers. The wearable device 9100 can also be similar to the wearable device 4100 in that it can be without a frame (e.g., the body portion 9102 may not include a frame within cavity 9109). In such implementations, the body portion 9102 can be configured to position and secure the electronic device 1400 without such a frame. For example, the body portion 9102 can include engagement features configured to retain and/or position the electronic device 1400 within the wearable device 9100 (e.g., at least partially within cavity 9109). As shown in FIG. 22E, such engagement features can include an upper ridge 9103a, a lower ridge 9103c, and an inner portion 9103b spanning between the upper ridge 9103a and the lower ridge 9103c. Such engagement features can be similar or the same and/or include any or all functionality as the engagement features of wearable device 4100 such as upper ridge 4103a, lower ridge 4103a, and inner portion 4103b. In some variants, the wearable device 9100 can include a frame similar or identical to the frame 1103 of wearable device 1100.

The wearable device 9100 can differ from the wearable device 4100 in that it can have a different securement portion configuration. While the loop 9105, tongue 9106a, and straps 9104a, 9104b of the securement portion can be similar or the same as corresponding features of the wearable device 4100, the strap 9104a of the wearable device 9100 can have a loop 9107 without an opening equivalent to the opening 4701a, and the strap 9104b can be without a protrusion corresponding to protrusion 4107b.

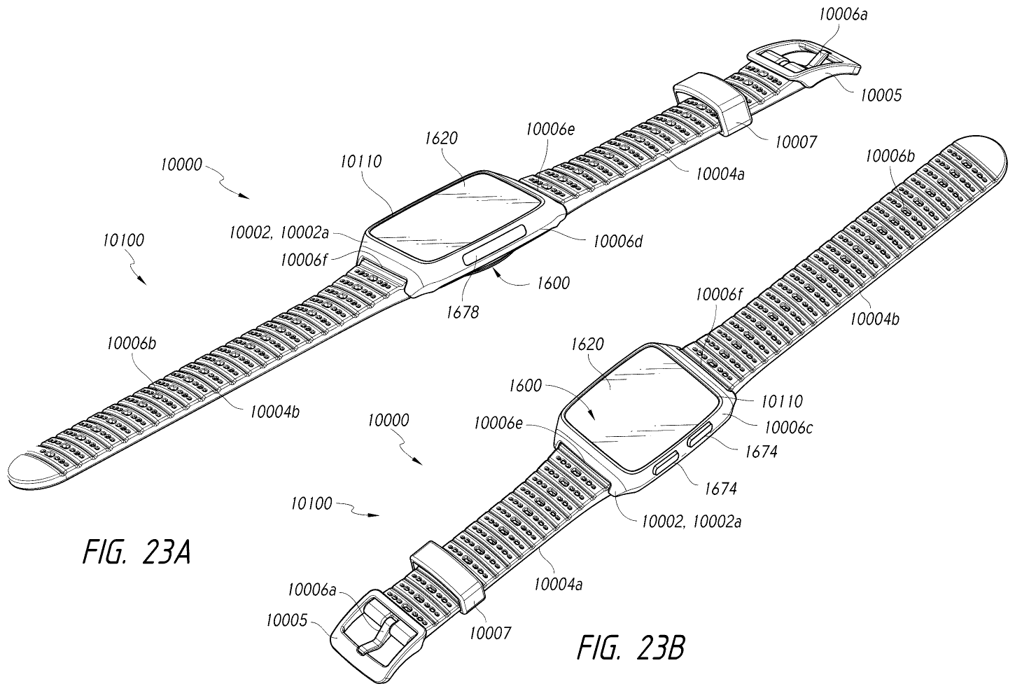

FIGS. 23A-23B illustrate a wearable system 10000 that is a variant of the wearable system 9000 described and illustrated with respect to FIGS. 22A-22E. FIGS. 23A-23B illustrate top perspective views of the wearable system 10000. The wearable system 10000 can be similar to the wearable system 9000 in some or many respects. The wearable system 10000 differs from the wearable system 9000 in that it can include and/or secure an electronic device 1600 that can include a display 1620 similar or identical to the display of the wearable sensor described in U.S. Pat Pub. No. 2021/0290120, U.S. Pat. Pub. No. 2023/0028745, and/or U.S. Provisional Application No. 63/386,474 incorporated by reference herein, which can be configured to display a plurality of physiological parameters monitored and/or determined by the wearable system 10000 and/or other information (e.g., such as the time, date, etc.). Further, the display 1620 can be configured to receive input from the subject, such as touch input, for interacting with the wearable system 10000. Such display 1620 can be disposed on a top of the electronic device 1600 such that it can be accessible when the wearable system 10000 is worn by a subject. The wearable system 10000 (e.g., the electronic module 1600) can also differ from the wearable system 9000 in that it can include an ECG electrode 1678 disposed along a side the electronic device 1600 such as shown in FIG. 23A (e.g., accessible via an opening at end 10006d of the wearable device). The wearable system 10000 (e.g., the electronic module 1600) can also differ from the wearable system 9000 by including one or more user interfaces 1674 similar or identical to the user interfaces 13 (e.g., user interface 13a and/or user interface 13b) of the wearable sensor described in U.S. Pat Pub. No. 2021/0290120, U.S. Pat. Pub. No. 2023/0028745, and/or U.S. Provisional Application No. 63/386,474 incorporated by reference herein, which can be configured as button(s). As shown in FIG. 23B, such user interfaces 1674 can be disposed along a side of the electronic device 1600 (e.g., accessible via opening(s) at end 10006c of the wearable device).

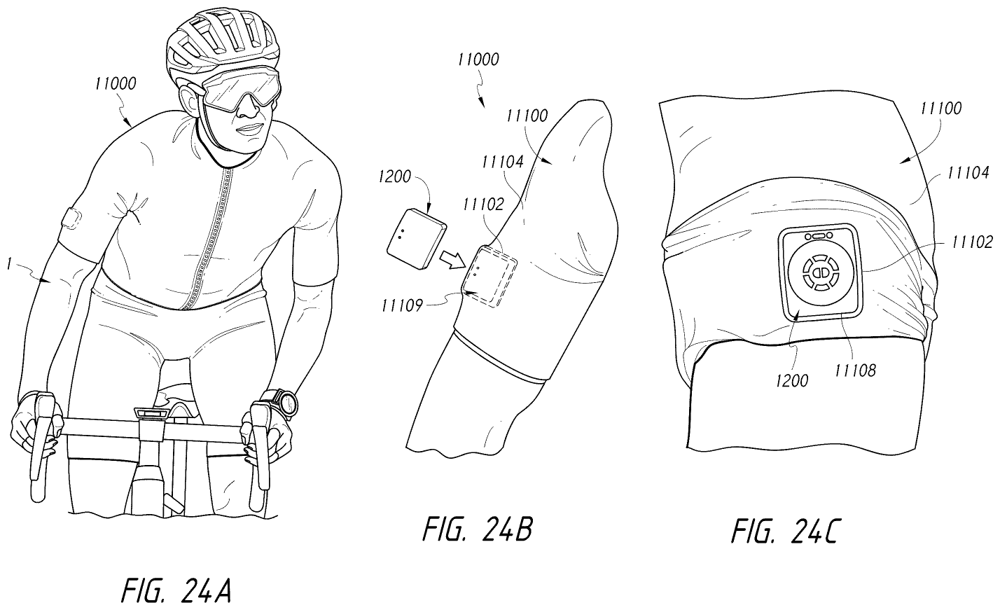

FIGS. 24A-24C illustrate another implementation of a wearable system 11000. The wearable system 11000 can include the electronic device 1200 and a wearable device 11100. The wearable device 11100 can be configured as a garment that can operably position the electronic device 1200 for measuring and/or monitoring at least one physiological parameter of the subject 1 as described herein. As shown in FIGS. 24A-24C, in some implementations the wearable device 11100 can secure to an upper body of the subject 1. The wearable device 11100 can include a body portion 11102 and a securement portion 11104, which can each include any of the features and/or functionality of any of the wearable devices described herein. The securement portion 11104 of the wearable device 11100 can be configured as a shirt that can be worn by the subject 1. The body portion 11102 can connect to the securement portion 11104, for example, at a body-facing side of the securement portion 11104. In some implementations, the wearable device 11100 and the electronic device 1200 of the wearable system 11000 can form a unitary structure. In some implementations, the electronic device 1200 can be removably connectable to the wearable device 11100.

The body portion 11102 of the wearable device 11100 can include a first side and a second side opposite the first side. Such second side can face toward the subject (e.g., face toward a tissue site of the subject) when the wearable device 11100 is worn by the subject. Furthermore, such second side can include an opening 11108 and a cavity 11109 that can be similar or the same and/or include any of the features and/or functionality of any of the wearable devices described herein. For example, the opening 11108 can be configured to allow the electronic device 1200 to be at least partially inserted therethrough and at least partially positioned within the cavity 11109. The body portion 11102 can comprise a flexible, stretchy, and/or resilient material configured to receive and operably position the electronic device 1200. For this, in some implementations, the body portion 11102 can include a frame similar or identical to any of the frames illustrated and/or described herein.

As shown in at least FIG. 24A, the wearable device 11100 can be configured to cover the electronic device 1200 such that the electronic device 1200 is not visible when the securement portion 11104 secures the body portion 11102 to the subject. In some implementations, at least a portion of the wearable device 11100 can comprise an opaque and/or non transparent material. In some implementations, the wearable device 11100 comprises a fabric material.

In some implementations, the wearable device 11100 can include features and/or materials to enhance a grip of the wearable device 11100 onto skin of the subject to prevent the electronic device 1200 from slipping or moving along the tissue site of the subject. Such features can include bumps, a roughened surface texture, an adjustable draw string, and adjustable band, and the like. Such materials can include a tacky and/or rubber-like material (e.g., a silicone and/or silicone rubber), an adhesive material, or the like. In some implementations, such features can comprise such materials. Furthermore, such features and/or materials can be disposed at least partially along a body-contacting side of the wearable device 11100. For example, such features and/or materials can be disposed at, adjacent, and/or around where the body portion 11102 connects to the securement portion 11104. In some implementations, the securement portion 11104 can include a compression shirt, a stretchy athletic shirt, or the like that can aid in securing the electronic device 1200 to the subject such that the electronic device 1200 is prevented from slipping and/or moving along a tissue site of the subject. In implementations where the securement portion 11104 is a shirt, such shirt can be short-sleeved, mid-sleeved, or long-sleeved.

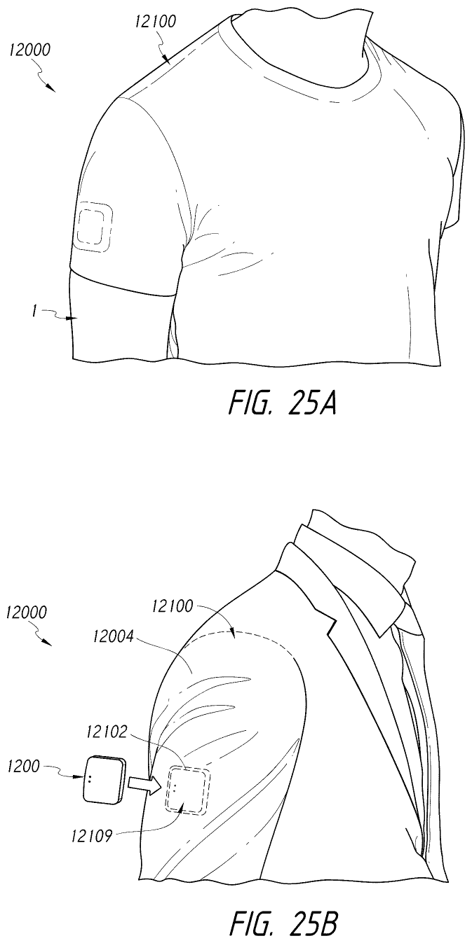

FIGS. 24A-24B illustrate a wearable system 12000 that is a variant of the wearable system 11000 described and illustrated with respect to FIGS. 24A-24C. The wearable system 12000 can be similar or the same as the wearable system 11000 in some, many, or all respects and can include any and/or all features and/or functionality of the wearable system 11000. As shown in FIG. 25A, the wearable system 12000 can have a wearable device 12100 configured as a shirt (e.g., such as an undershirt). As such, the wearable system 12000 can be configured to be worn either by itself on an upper body of the subject, and/or under other garments of the subject such as shown in FIG. 25B (where such wearable system 12000 is worn underneath a dress shirt and jacket). The wearable system 12000 can include the electronic device 1200 and the wearable device 12100. The wearable device 12100 can include a body portion 12102 and a securement portion 12004 that can be the same or similar to the body portion 11102 and securement portion 11004 of the wearable device 11100. For example, the body portion 12102 can include a cavity 12109 for receiving and operably positioning at least a portion of the electronic device 1200.

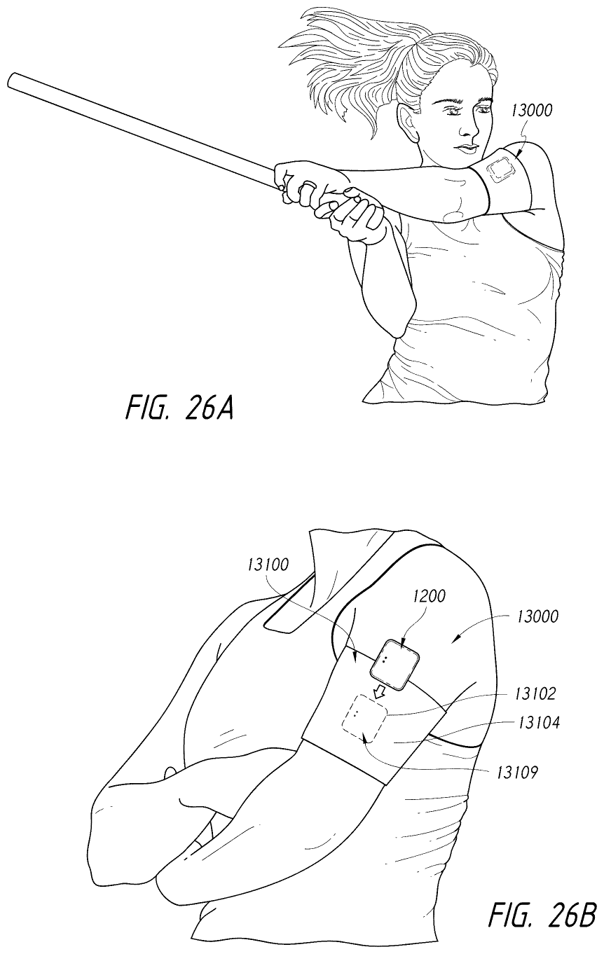

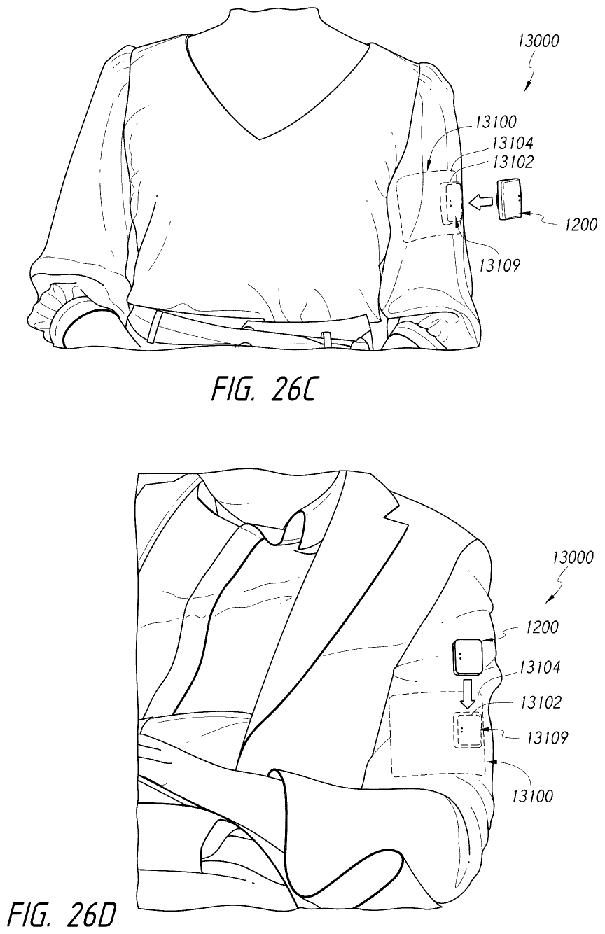

FIGS. 26A-26D illustrate another implementation of a wearable system 13000. The wearable system 13000 can include the electronic device 1200 and a wearable device 13100. The wearable device 13100 can be configured as a band that can operably position the electronic device 1200 for measuring and/or monitoring at least one physiological parameter of the subject 1 as described herein. As shown in FIGS. 26A-26D, in some implementations the wearable device 13100 can secure to a portion of an upper body of the subject 1 (e.g., an upper arm of the subject). The wearable device 13100 can include a body portion 13102 and a securement portion 13104, which can each include any of the features and/or functionality of any of the wearable devices described herein. The securement portion 13104 of the wearable device 13100 can be configured as a band that can be worn by the subject 1. In some implementations, the wearable device 13100 can be integrated within other garments (e.g., such as a dress or jacket among others as shown in FIGS. 26C-26D) and/or the wearable system 13000 can be worn underneath other garments. The body portion 13102 can connect to the securement portion 13104, for example, at a body-facing side of the securement portion 13104. In some implementations, the wearable device 13100 and the electronic device 1200 of the wearable system 13000 can form a unitary structure. In some implementations, the electronic device 1200 can be removably connectable to the wearable device 13100.

The body portion 13102 of the wearable device 13100 can include a first side and a second side opposite the first side. Such second side can face toward the subject (e.g., face toward a tissue site of the subject) when the wearable device 13100 is worn by the subject. Furthermore, such second side can include an opening and a cavity 13109 that can be similar or the same and/or include any of the features and/or functionality of any of the wearable devices described herein. For example, the opening can be configured to allow the electronic device 1200 to be at least partially inserted therethrough and at least partially positioned within the cavity 13109. The body portion 13102 can comprise a flexible, stretchy, and/or resilient material configured to receive and operably position the electronic device 1200. For this, in some implementations, the body portion 13102 can include a frame similar or identical to any of the frames illustrated and/or described herein.

As shown in at least FIG. 26B, the wearable device 13100 can be configured to cover the electronic device 1200 such that the electronic device 1200 is not visible when the securement portion 13104 secures the body portion 13102 to the subject. In some implementations, at least a portion of the wearable device 13100 can comprise an opaque and/or non transparent material. In some implementations, the wearable device 13100 comprises a fabric material.

In some implementations and as shown, the securement portion 1304 can extend beyond ends of the body portion 13102 (e.g., be wider than a width of the body portion 13102) to aid in securing the electronic device 1200 to a tissue site of the subject to prevent slipping or moving of the electronic device 1200 relative to such tissue site. In some implementations, the wearable device 13100 can include features and/or materials to enhance a grip of the wearable device 13100 onto skin of the subject to prevent the electronic device 1200 from slipping or moving along the tissue site of the subject. Such features can include bumps, a roughened surface texture, an adjustable draw string, and adjustable band, and the like. Such materials can include a tacky and/or rubber-like material (e.g., a silicone and/or silicone rubber), an adhesive material, or the like. In some implementations, such features can comprise such materials. Furthermore, such features and/or materials can be disposed at least partially along a body-contacting side of the wearable device 13100. For example, such features and/or materials can be disposed at, adjacent, and/or around where the body portion 13102 connects to the securement portion 13104. In some implementations, the securement portion 11104 can include a compression band, a stretchy athletic band, or the like that can aid in securing the electronic device 1200 to the subject such that the electronic device 1200 is prevented from slipping and/or moving along a tissue site of the subject.

Although examples and certain orientations and configurations of various aspects of the wearable systems described in this disclosure have been provided, alternative orientations and configurations for such aspects are to be considered included as a part of this disclosure. For example, although certain orientations and configurations for the orientation/direction in which the wearable systems described herein can be secured to a subject have been provided, the wearable systems described herein can be secured to a subject in any orientation/direction. Furthermore, the wearable systems described herein can be adapted for securement to various portions of a subject's body as described herein.

The wearable systems described herein and any variations thereof and/or any of their components can be configured to be waterproof, water resistant, drip proof, shock proof, dust proof, and/or dust resistant. While the wearable systems have been described as having a rechargeable battery, the battery can be nonrechargeable or single use. In some implementations, a battery of the wearable system can be rechargeable but non-removable from the system. In such a case, the wearable system can include a charge port configured to receive a power cable for charging. In some variants, an electronic device of any of the wearable systems described herein can be permanently connected to a wearable device of any of the wearable systems described herein.

In some implementations, any or all of the components of the wearable devices described herein can be configured to be reusable (which may also be referred to herein as “durable”), refurbishable, reprocessible, or the like. In such a case, any or all components can be sanitized between uses and/or between subjects. In some implementations, all components of the wearable systems as described herein can be configured to be reusable except for the wearable devices described herein. In some implementations, one or more components of the wearable systems as described herein, or any portions thereof, can be configured as single use or for a limited number of uses (which may be referred to herein as “disposable”).

Any of the features and/or functionality of the wearable systems and their components described herein can be implemented in another of the wearable systems described herein. For example, any of the wearable devices described herein can include any of the body portion configurations and/or securement portion configurations of another wearable device described herein. As another example, any of the wearable systems can be adapted to receive any one of the electronic devices described herein. Furthermore, any of the features and/or functionality of the wearable systems and their components described herein can be omitted in another of the wearable systems described herein. For example, any of the openings in a first side of the wearable devices described herein can be omitted (e.g., if using an electronic device such as 1200, 1300 that may not have an ECG electrode on a top thereof).

### Additional Considerations and Terminology

Certain categories of persons, such as caregivers, clinicians, doctors, nurses, and friends and family of a subject, may be used interchangeably to describe a person providing care to the subject. Furthermore, subjects, patients, or users used herein interchangeably refer to a person who is wearing a sensor or is connected to a sensor or whose measurements are used to determine a physiological parameter or a condition. Parameters may be, be associated with, and/or be represented by, measured values, display icons, alphanumeric characters, graphs, gauges, power bars, trends, or combinations. Real time data may correspond to active monitoring of a subject, however, such real time data may not be synchronous to an actual physiological state at a particular moment. Measurement value(s) of a parameter such as any of those discussed herein, unless specifically stated otherwise, or otherwise understood with the context as used is generally intended to convey a measurement or determination that is responsive to and/or indicative of the physiological parameter.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more implementations necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the implementation, certain acts, events, or functions of any of the processes or algorithms described herein can be performed in a different sequence, can be added, merged, or left out altogether (for example, not all described operations or events are necessary for the practice of the algorithm). Moreover, in certain implementations, operations or events can be performed concurrently, e.g., through multi-threaded processing, interrupt processing, or multiple processors or processor cores or on other parallel architectures, rather than sequentially.

Various illustrative logical blocks, modules, routines, and algorithm steps that may be described in connection with the disclosure herein can be implemented as electronic hardware (e.g., ASICs or FPGA devices), computer software that runs on general purpose computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the disclosure herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. A processor can include an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A wearable system comprising:
an electronic device configured to measure one or more physiological parameters of a subject, the electronic device comprising at least one light emitter and at least one light detector and is configured to measure at least a pulse oximetry measurement; and
a wearable device configured to cover and position the electronic device, the wearable device comprising:
a body portion comprising a first side, a second side opposite the first side, a cavity, and an opening in the second side configured to allow the electronic device to be at least partially inserted through said opening and at least partially positioned within said cavity; and
a securement portion connected to the body portion and configured to secure said body portion to the subject to prevent the electronic device from slipping or moving along a tissue site of the subject;
wherein said second side of said body portion is configured to face toward said tissue site of the subject when the wearable device is secured to the subject; and
wherein the wearable device covers the electronic device such that the electronic device is not visible when the securement portion secures the body portion to the subject.

2. The wearable system of claim 1, wherein the electronic device further comprises:
at least one ECG electrode and is configured to measure at least an ECG measurement; and
a communication module configured to facilitate wireless communication with a separate device.

3. The wearable system of claim 1, wherein the electronic device comprises a battery and a charge port, and wherein the charge port is accessible for charging the battery of the electronic device when the electronic device is positioned at least partially within said cavity.

4. The wearable system of claim 1, wherein said securement portion comprises a strap, a band, or a garment.

5. The wearable system of claim 1, wherein said first side of said body portion does not comprise an opening.

6. The wearable system of claim 1, wherein said body portion comprises a flexible, stretchy, and/or resilient material configured to allow a size of said opening to be increased to allow the electronic device to be at least partially inserted through said opening and at least partially positioned within said cavity.

7. The wearable system of claim 1, wherein said body portion further comprises a frame positioned within said cavity configured to position the electronic device.

8. The wearable system of claim 7, wherein said frame comprises a resilient material.

9. The wearable system of claim 1, wherein said body portion and said securement portion are seamlessly integrated with one another.

10. The wearable system of claim 9, wherein said body portion and said securement portion comprise a first layer, a second layer, and a third layer, said second layer disposed in between said first and third layers, wherein said first side of the body portion comprises said first and second layers, and wherein said second side of the body portion comprises said third layer.

11. The wearable system of claim 10, wherein said first and third layers comprise a fabric material, and wherein said second layer comprises an unbroken loop fabric material.

12. A wearable device configured to cover and position an electronic device configured to measure a physiological parameter of a subject, the wearable device comprising:
a body portion comprising a first side, a second side opposite the first side, a cavity, and an opening in the second side configured to allow the electronic device to be at least partially inserted through said opening and at least partially positioned within said cavity; and
a securement portion connected to the body portion and configured to secure said body portion to the subject to prevent the electronic device from slipping or moving along a tissue site of the subject;
wherein said second side of said body portion is configured to face toward said tissue site of the subject when the wearable device is secured to the subject; and
wherein the wearable device covers the electronic device such that the electronic device is not visible when the securement portion secures the body portion to the subject.

13. The wearable device of claim 12, wherein said securement portion comprises a strap, a band, or a garment.

14. The wearable device of claim 12, wherein said first side of said body portion does not comprise an opening.

15. The wearable device of claim 12, wherein said body portion comprises a flexible, stretchy, and/or resilient material configured to allow a size of said opening to be increased to allow the electronic device to be at least partially inserted through said opening and at least partially positioned within said cavity.

16. The wearable device of claim 12, wherein said body portion further comprises a frame positioned within said cavity configured to position the electronic device.

17. The wearable device of claim 16, wherein said frame comprises a resilient material.

18. The wearable device of claim 12, wherein said body portion and said securement portion are seamlessly integrated with one another.

19. The wearable device of claim 18, wherein said body portion and said securement portion comprise a first layer, a second layer, and a third layer, said second layer disposed in between said first and third layers, wherein said first side of the body portion comprises said first and second layers, and wherein said second side of the body portion comprises said third layer.

20. The wearable device of claim 19, wherein said first and third layers comprise a fabric material, and wherein said second layer comprises an unbroken loop fabric material.
